(19) United States
(12) Patent Application Publication
(10) Pub. No.: US 2009/0286225 A1
(43) Pub. Date: Nov. 19, 2009

(54) METHOD AND APPARATUS FOR BACTERIOPHAGE-BASED DIAGNOSTIC ASSAYS

(75) Inventors: John H. Wheeler, Boulder, CO (US); Michael Fiechtner, Poway, CA (US); Jonathan D. Smith, Boulder, CO (US); Duane Bush, Fort Collins, CO (US); Alene A. Campbell, Boulder, CO (US); Breanna C. Smith, Lyons, CO (US)

Correspondence Address: PATTON BOGGS LLP, 1801 CALFORNIA STREET, SUITE 4900, DENVER, CO, 80202, US

(73) Assignee: MicroPhageTM Incorporated, Longmont, CO (US)

(21) Appl. No.: 12/402,337

(22) Filed: Mar. 11, 2009

Related U.S. Application Data
(63) Continuation-in-part of application No. 12/346,656, filed on Dec. 30, 2008.
Application No. 12/346,656 is a division of application No. 10/823,294, filed on Apr. 12, 2004, now abandoned.
Application No. 10/823,294 is a continuation-in-part of application No. 11/933,083, filed on Oct. 31, 2007.
Application No. 11/933,083 is a continuation-in-part of application No. PCT/US08/66962, filed on Jun. 13, 2008.
(60) Provisional application No. 60/544,437, filed on Feb. 13, 2004.
(60) Provisional application No. 60/557,962, filed on Mar. 31, 2004.
(60) Provisional application No. 60/855,648, filed on Oct. 31, 2006.
(60) Provisional application No. 60/860,839, filed on Nov. 22, 2006.
(60) Provisional application No. 60/934,781, filed on Jun. 15, 2007.
(60) Provisional application No. 61/085,068, filed on Jul. 31, 2008.
(60) Provisional application No. 61/097,626, filed on Sep. 17, 2008.

(51) Int. Cl. C12Q 1/70; C12M 1/00

(52) U.S. Cl. 435/5; 435/287.7; 435/287.2; 435/303.1

(57) ABSTRACT

Bacteriophage are combined with a test sample in an incubator, and the bacteriophage-exposed test sample is conjugated and applied to a sample pad in contact with a lateral flow strip to determine the presence or absence of a target bacterium. The conjugation may be performed in the sample pad or prior to application of the bacteriophage-exposed test sample to the pad. The incubator comprises a bacteriophage container and an incubation container separated by a valve. The test sample may be inserted into the incubation chamber using a swab or a rod with a piercing tip and a sample collection eye. The valve comprises a breakable stem. An antibiotic may be added to the test sample to determine the antibiotic resistance or susceptibility of the bacterium.

### CROSS-REFERENCE TO RELATED APPLICATIONS

This application is a Continuation-in-part of U.S. patent application Ser. No. 12/346,656 filed on Dec. 30, 2008 which is a divisional application of U.S. patent application Ser. No. 10/823,294 filed on Apr. 12, 2004, and published as US Patent Application Publication No. 2005/0003346 on Jan. 6, 2005, which claims the benefit of U.S. Provisional Application No. 60/544,437 filed on Feb. 13, 2004, and U.S. Provisional Application No. 60/557,962 filed on Mar. 31, 2004. This application is a Continuation-in-part of U.S. patent application Ser. No. 11/933,083 filed on Oct. 31, 2007, which claims the benefit of U.S. Provisional Application 60/855,648 filed on Oct. 31, 2006, and U.S. Provisional Application No. 60/860,839 filed on Nov. 22, 2006. This application is a Continuation-in-part of PCT Application No. PCT/US08/66962 filed on Jun. 13, 2008, which claims the benefit of U.S. Provisional Application 60/934,781 filed on Jun. 15, 2007. This application claims the benefit of U.S. Provisional Application No. 61/085,068 filed on Jul. 31, 2008. This application claims the benefit of U.S. Provisional Application No. 61/097,626 filed on Sep. 17, 2008. All of the above patent applications, both provisional and non-provisional, are hereby incorporated by reference to the same extent as though fully contained herein.

### FIELD OF THE INVENTION

The invention relates generally to the field of identification of microscopic living organisms, and more particularly to the identification of microorganisms using bacteriophage.

### BACKGROUND OF THE INVENTION

Currently, bacteria that may be causing an infection or other health problems are identified by bacteria culture methods. Generally, it takes a day or several days to grow sufficient bacteria to enable the detection and identification of the bacteria. By that time, the person or persons infected by the bacteria may be very sick or dead. Thus, there is a need for more rapid detection and identification of bacteria. Further, when bacteria infection is suspected, a physician will often prescribe a broad spectrum antibiotic. This has led to the development of antibiotic-resistant bacteria, which has further enhanced the need for more rapid detection of bacteria. Because of these issues, infection of patients in hospitals by methicillin-resistant staphophyloccosus aureus (MRSA), for example, has become endemic. A 2007 report in Emerging Infectious Diseases, a publication of the Centers for Disease Control and Prevention (CDC), estimated that the number of MRSA infections treated in hospitals doubled nationwide, from approximately 127,000 in 1999 to 278,000 in 2005, while at the same time deaths increased from 11,000 to more than 17,000. Another study led by the CDC and published in the Oct. 17, 2007 issue of the Journal of the American Medical Association estimated that MRSA would have been responsible for 94,360 serious infections and associated with 18,650 hospital stay-related deaths in the United States in 2005. These figures suggest that MRSA infections are responsible for more deaths in the US each year than AIDS. See http://en.wikipedia.org/wiki/Methicilin-resistant_{—}Staphylococcus_{—}aureus.

The method and apparatus of the invention utilize bacteriophage, or simply phage, to indirectly detect the presence of target microscopic living organisms in a sample. In this disclosure, the terms “bacteriophage” and “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, and other microscopic living organisms and uses them to replicate itself. Here, “microscopic” means that the largest dimension is one millimeter or less. Bacteriophage are organisms that have evolved in nature to use bacteria as a means of replicating themselves. A phage does this by attaching itself to a bacterium and injecting its DNA into that bacterium, inducing it to replicate the phage hundreds or even thousands of times. Some bacteriophage, called lytic bacteriophage, rupture the host bacterium, releasing the progeny phage into the environment to seek out other bacteria. The total incubation time for phage infection of a bacterium, phage multiplication, or amplification in the bacterium to lysing of the bacterium takes anywhere from tens of minutes to hours, depending on the phage and bacterium in question and the environmental conditions. The progeny phage then infect the bacteria again, and replicate hundreds or even thousands of times again, and so on. This is called “phage amplification”. If a relatively small concentration of bacteriophage are introduced into a sample, and a few hours later the phage have amplified, this is an indirect indication that bacteria were present. If the phage introduced into the sample infect only a particular type of bacteria, that is, the phage are specific to the particular type of bacterium, then the amplification of the phage is an indirect indication of the presence of the particular bacterium. Since the number of phage that are present after amplification is millions of times larger than the number of bacteria, at least in theory, the phage should be easier to detect than the bacteria themselves. See, for example, U.S. Pat. No. 5,985,596 issued Nov. 16, 1999 and No. 6,461,833 B1 issued October 8, both to Stuart Mark Wilson; U.S. Pat. No. 4,861,709 issued Aug. 29, 1989 to Ulitzur et al.; U.S. Pat. No. 5,824,468 issued Oct. 20, 1998 to Scherer et al.; U.S. Pat. No. 5,656,424 issued Aug. 12, 1997 to Jurgensen et al.; U.S. Pat. No. 6,300,061 B1 issued Oct. 9, 2001 to Jacobs, Jr. et al.; U.S. Pat. No. 6,555,312 B1 issued Apr. 29, 2003 to Hiroshi Nakayama; U.S. Pat. No. 6,544,729 B2 issued Apr. 8, 2003 to Sayler et al.; U.S. Pat. No. 5,888,725 issued Mar. 30, 1999 to Michael F. Sanders; U.S. Pat. No. 6,436,652 B1 issued Aug. 20, 2002 to Cherwonogrodzky et al.; U.S. Pat. No. 6,436,661 B1 issued Aug. 20, 2002 to Adams et al.; U.S. Pat. No. 5,498,525 issued Mar. 12, 1996 to Rees et al.; Angelo J. Madonna, Sheila VanCuyk, and Kent J. Voorhees, “Detection Of Esherichia Coli Using Immunomagnetic Separation And Bacteriophage Amplification Coupled With Matrix-Assisted Laser Desorption/Ionization Time-Of-Flight Mass Spectrometry”, Wiley InterScience, DOI:10.1002/rem.900, 24 Dec. 2002; and US Patent Application Publication No. 2004/0224359 published Nov. 11, 2004.

While detection and identification of bacteria using bacteriophage amplification is theoretically possible, in practice, it does not work well outside the laboratory because the bacteriophage amplification process is complex and many things can interfere with it. Bacteria have developed defenses, bacteriophage are everywhere, and undesirable species of bacteriophage can easily contaminate a sample, and many other practical problems have prevented bacterial detection and identification using bacteriophage from becoming commercially successful, even though the idea has been around for a generation and huge sums have been invested in trying to make the process work. In particular, the bacteria identification and detection methods in the above references have disadvantages that impede their commercial usefulness. The methods of the latter two references require a multimillion dollar MALDI spectrometer, which make it impractical. All of the prior art references require one or more complicated laboratory procedures that take days; thus, the cost is high and the whole reason for the proposals for a bacteriophage-based assay—its speed—is vitiated. A method and apparatus for detection of bacteria using bacteriophage that effectively utilized the potential speed advantage of the bacteriophage detection and identification method in a real environment, therefore, is desirable. If this method and apparatus were also low cost, it would be highly desirable. In addition, if the method could at the same time determine the resistance of the bacteria to specific antibiotics, such a method and apparatus would be a great boon to diagnostic medicine.

### BRIEF SUMMARY OF THE INVENTION

The invention solves the above problems, as well as other problems of the prior art, by providing, for the first time, a system that can rapidly detect and identify specific bacteria and which can be performed reliably by health practitioners without specialized training. The invention provides a system that permits the bacterial detection and identification process to be accurately and competently performed by conventional personnel in hospitals, clinics, and physician's offices, without the need for specially trained laboratory personnel. The system of the invention can also be employed for quickly determining antibiotic resistance and susceptibility of the bacteria, also by conventionally trained health professionals.

The invention provides a method for determining the presence or absence of a target bacterium in a test sample, the method comprising: combining a bacteriophage specific to the target bacteria with the sample; incubating the sample sufficiently to permit the bacteriophage to infect the target bacteria and to multiply in the target bacteria to create progeny bacteriophage to create a bacteriophage exposed sample; providing a sample pad and a porous strip, the sample pad in contact with the porous strip, the porous strip having a fixation substance embedded in the porous strip at a fixation area, the fixation substance capable of attaching to either the bacteriophage or the bacteriophage conjugate; applying the progeny bacteriophage to the sample pad; conjugating the progeny bacteriophage to a bacteriophage conjugate to form a bacteriophage/conjugate complex; flowing the bacteriophage/conjugate through the porous strip to the fixation area; and determining the presence or absence of a sufficient amount of fixated bacteriophage/conjugate complex at the fixation area to determine the presence or absence of the target bacterium. Preferably, the sample pad contains the conjugate and the conjugating is performed in the sample pad. Preferably, the conjugating is performed prior to the applying. Preferably, the conjugating comprises combining the conjugate and the progeny bacteriophage in an incubation container. Preferably, the porous strip includes a conjugate area and the conjugating is performed in the test strip. Preferably, the providing a porous strip comprises embedding an antibody in the fixation area. Preferably, the providing comprises embedding a control fixation substance in a control area in the porous strip, the control fixation substance comprising a substance capable of attaching to the conjugate.

The invention also provides a system for determining the presence or absence of a target bacterium in a test sample, the system comprising: a bacteriophage specific to the bacterium; a detectable conjugate to the bacteriophage; a sample pad for receiving a test sample that may contain the target bacterium; and a porous strip in contact with the sample pad and having a fixation area comprising a test fixation substance embedded in the porous strip, the test fixation substance comprising a substance capable of attaching to either the bacteriophage or the conjugate. Preferably, the conjugate is embedded in the sample pad. Preferably, the system further includes a bacteriophage incubation container containing the conjugate. Preferably, the system also includes a strip holder enclosing the porous strip, the strip holder having a window exposing the fixation area and labeling adjacent the window indicating the approximate location of the fixation area. Preferably, the test fixation substance comprises an antibody to the bacteriophage. Preferably, the porous strip further includes a conjugate area in which the conjugate is embedded, the conjugate area located in the porous strip between the sample pad and the fixation area. Preferably, the system further comprises a control area comprising a control fixation substance, the control fixation substance comprising a substance capable of attaching to the conjugate. Preferably, the bacteriophage comprises a plurality of different types of bacteriophage, each of the different bacteriophage being specific to the target bacterium.

The invention also provides a kit for determining the presence or absence of a target bacterium in a sample to be tested, the kit comprising: a bacteriophage; a bacteriophage incubation container having an opening for inserting a sample containing the target bacterium; and a substrate at least a portion of which changes color if a predetermined amount of either the bacteriophage or a biological substance associated with the bacteriophage is present. Preferably, the kit further includes a bacteriophage container containing the bacteriophage in a buffer solution. Preferably, the kit further includes a connector for connecting the bacteriophage container to the incubation container with a fluid tight seal. Preferably, the kit further includes a buffer solution in the bacteriophage container, the buffer solution also containing a substance that enhances bacteriophage amplification, the substance being different than the buffer solution. Preferably, the buffer solution also contains a substance that inhibits replication of the bacteriophage in potentially cross-reactive, non-target bacteria. Preferably, the substrate comprises a porous strip. Preferably, the kit further includes a dropper for applying a fluid containing the target bacterium and the bacteriophage to the porous strip. Preferably, the bacteriophage container contains a conjugate for the bacteriophage. Preferably, the incubation container contains a conjugate for the bacteriophage. Preferably, the bacteriophage comprises a plurality of different types of bacteriophage, each of the different bacteriophage being specific to the target bacterium.

The invention further provides a bacteriophage incubator comprising: a bacteriophage incubation container; and a bacteriophage incubation fluid container containing bacteriophage incubation fluid comprising bacteriophage; said bacteriophage incubator characterized by: said incubation container and said incubation fluid container each having a connection portion formed to permit said incubation container to be connected to said incubation fluid container with a fluid tight seal; said incubator further comprising: a valve located between said incubation fluid container and said incubation container when said incubation fluid container is connected to said incubation container, said valve having a closed condition in which said incubation fluid container does not fluidly communicate with said incubation container and an open position in which said fluid container fluidly communicates with said incubation container. Preferably, said valve comprises a breakable stem. Preferably, said bacteriophage incubator further comprises a collector adapted for insertion into said incubation container. Preferably, said collector comprises a swab. Preferably, said collector comprises a rod having a piercing tip and an eye for receiving sample fluid. Preferably, said incubation fluid container comprises a flexible bulb. Preferably, said incubator includes an applicator tip communicating with said incubation container. Preferably, said incubation fluid container comprises a flexible bulb, and said incubator further comprises a tube connecting said applicator tip and said flexible bulb. Preferably, said incubator further includes a filter between said incubation container and said applicator tip. Preferably, said incubator further comprises a porous member containing an incubation reagent, said porous member located within said incubation container. Preferably, said incubation reagent comprises an antibiotic. Preferably, said incubation reagent comprises a bacteriophage conjugate. Preferably, said bacteriophage incubation fluid comprises a bacteriophage conjugate. Preferably, said incubation fluid comprises a base broth and a substance that enhances bacteriophage amplification dissolved in said base broth, said substance being different than said base broth. Preferably, said incubation fluid further includes a substance that inhibits replication of said bacteriophage in potentially cross-reactive, non-target bacteria.

The invention also provides a method of determining the presence or absence of a target bacteria in a sample to be tested, said method comprising: combining with said sample an amount of parent bacteriophage capable of attaching to said target bacteria to create a bacteriophage-exposed sample; providing conditions to said bacteriophage-exposed sample sufficient to allow said bacteriophage to attach to said target bacteria to provide a detectable amount of either bacteriophage or a biological substance associated with said bacteriophage in a bacteriophage-exposed sample; and assaying said bacteriophage-exposed sample to detect the presence or absence of said bacteriophage or said biological substance associated with said bacteriophage to determine the presence or absence of said target bacteria, said method characterized by said combining comprising: providing a collection/incubation system comprising a bacteriophage container containing a fluid comprising said bacteriophage and an incubation container; collecting said bacteria with a collector; placing said collector in said incubating container; connecting said bacteriophage container to said incubation container, said connecting providing a fluid tight seal; and, without breaking said fluid tight seal, opening said valve to permit said bacteriophage fluid to flow into said incubation container. Preferably, said method further comprises applying said bacteriophage-exposed sample to a porous strip. Preferably, said method further comprises conjugating said bacteriophage or said biological substance to a conjugate capable of attaching to said bacteriophage or said biological substance. Preferably, said method further comprises applying said bacteriophage-exposed sample to a porous strip. Preferably, said conjugating comprises conjugating said conjugate to said bacteriophage prior to said applying. Preferably, said conjugating comprises conjugating said conjugate to said bacteriophage after said applying. Preferably, said conjugating comprises attaching an antibody to said bacteriophage. Preferably, said conjugating comprises conjugating said bacteriophage antibody to a colored marker. Preferably, said providing comprises providing conditions to permit said bacteriophage to infect said target microorganism, to multiply in said target microorganism to produce progeny bacteriophage, and for said conjugate to attach to said progeny bacteriophage. Preferably, said assaying comprises capturing a bacteriophage-conjugate complex in a zone or line of immobilized antibodies. Preferably, said providing conditions comprises providing conditions to permit said bacteriophage to infect said target bacteria and to multiply in said target bacteria to create progeny bacteriophage, and said detecting comprises detecting said progeny bacteriophage. Preferably, said opening said valve comprises breaking a breakable stem separating said bacteriophage container and said incubation container. Preferably, said bacteriophage container comprises a flexible bulb, and said combining further comprises compressing said flexible bulb. Preferably, said assaying comprises applying the fluid in said incubation container to a porous strip, and said applying includes compressing said flexible bulb.

The invention further provides a method of determining the resistance or susceptibility of a target bacterium to an antibiotic, said method comprising: providing a first sample containing said target bacteria; adding said antibiotic to said first sample; combining said first sample with a bacteriophage capable of infecting said target bacteria to create a first bacteriophage-exposed sample; providing conditions to said first bacteriophage-exposed sample sufficient to allow said bacteriophage to infect said target bacterium and to multiply in said target bacterium to create a detectable amount of either said bacteriophage or a biological substance associated with said bacteriophage in said first bacteriophage-exposed sample; assaying said first bacteriophage-exposed sample to detect the presence or absence of an amount of said bacteriophage or said biological substance associated with said bacteriophage; and if said amount of said bacteriophage or biological substance is above an amount associated with a predetermined indicator, determining that said target bacteria is resistant to said antibiotic, and if said amount of said bacteriophage or biological substance associated with said bacteriophage is below a predetermined susceptibility indicator, determining that said target bacteria is susceptible to said antibiotic. Preferably, said assaying comprises applying said first bacteriophage-exposed sample to a first porous strip. Preferably, said method further comprises providing a second bacteriophage-exposed sample separate from said first bacteriophage-exposed sample; and said assaying further comprises applying said second bacteriophage-exposed sample to a second porous strip. Preferably, said adding comprises adding a plurality of said antibiotics to said first sample. Preferably, said combining comprises: providing a collection/incubation system comprising a bacteriophage container and an incubation container, said bacteriophage container containing said bacteriophage, said bacteriophage container and said incubation container separated by a valve; collecting said bacteria with a collector; placing said collector in said incubating container; and opening said valve to permit said bacteriophage to flow into said incubation container.

The invention, for the first time, provides a commercially useful method of using bacteriophage amplification to identify bacteria. The method and apparatus of the invention not only retain the speed of detection that has made bacterial identification using bacteriophage so promising, but also permit the identification process to be effectively carried out by conventional health care professionals without the need for complex and costly laboratory procedures and specially trained personnel. Numerous other features, objects, and advantages of the invention will become apparent from the following detailed description when read in conjunction with the accompanying drawings.

### BRIEF DESCRIPTION OF THE DRAWINGS

FIG. 1 is a perspective view illustrating a preferred embodiment of a bacteria identification and antibiotic susceptibility/resistance dual collection, incubation, and applicator system according to the invention;

FIG. 2 is a front perspective view of the dual system holder of FIG. 1;

FIG. 3 is a back perspective view of the dual system holder of FIG. 1;

FIG. 4 is an exploded view of the collection, incubation, and applicator system of FIG. 1;

FIG. 5 is a cross-sectional view of the collection, incubation, and applicator system of FIG. 4;

FIG. 6 is a top perspective view of a preferred embodiment of a bacteria identification antibiotic susceptibility/resistance dual lateral flow strip bacteria detection assembly according to the invention;

FIG. 7 is an exploded view of the dual lateral flow strip assembly of FIG. 6;

FIG. 8 is a top perspective view of an alternative embodiment of a bacteria identification assembly according to the invention;

FIG. 9 is a top perspective view of the bacteria identification assembly of FIG. 8 with the top cover opened;

FIG. 10 is a bottom perspective view of the bacteria identification assembly of FIG. 10 with the bottom cover open;

FIG. 11 is a perspective view of a preferred embodiment of an alternative embodiment of a sample collector according to the invention;

FIG. 12 is a perspective view of the sample collector of FIG. 11 inserted into a blood vial;

FIG. 13 is a cross-sectional view of an alternative embodiment of a collection, incubation, and applicator system according to the invention, including the collector of FIG. 11;

FIG. 14 is a perspective view of another preferred embodiment of a lateral flow strip assembly according to the invention;

FIG. 15 is an exploded top view of the flow strip assembly of FIG. 14;

FIG. 16 is an exploded bottom view of the flow strip assembly of FIG. 14;

FIG. 17 is an exploded view of another preferred embodiment of a collection, incubation, and applicator system according to the invention;

FIG. 18 is a cross-sectional view of the incubation and applicator system portion of the system of FIG. 17;

FIG. 19 illustrates the system of FIG. 17 showing how the collector is inserted into the incubator and the use of the applicator; and

FIG. 20 is a depiction of three photographs showing the results of an embodiment of the invention in which the conjugate is combined with the bacteriophage prior to application to a flow strip.

### DETAILED DESCRIPTION OF THE INVENTION

The method and apparatus of the invention utilize bacteriophage, or simply phage, to indirectly detect the presence of a target microscopic living organism in a sample, identify the organism, and determine whether the organism is susceptible or resistant to a specific antibiotic. The invention provides a bacteria identification and antibiotic susceptibility/resistance system that preferably includes a collection, incubation, and applicator system 10 in combination with a bacteria identification antibiotic susceptibility/resistance lateral flow strip 200. The bacteriophage described herein are specific to the target microorganism. It is understood that the term “specific” herein is a relative term, since no bacteriophage is one hundred percent specific to a target microorganism. Thus, herein stating that a bacteriophage is specific to a target microorganism means that at least seventy-five percent of the time that the bacteriophage attaches to a microorganism, it will attach to the target microorganism.

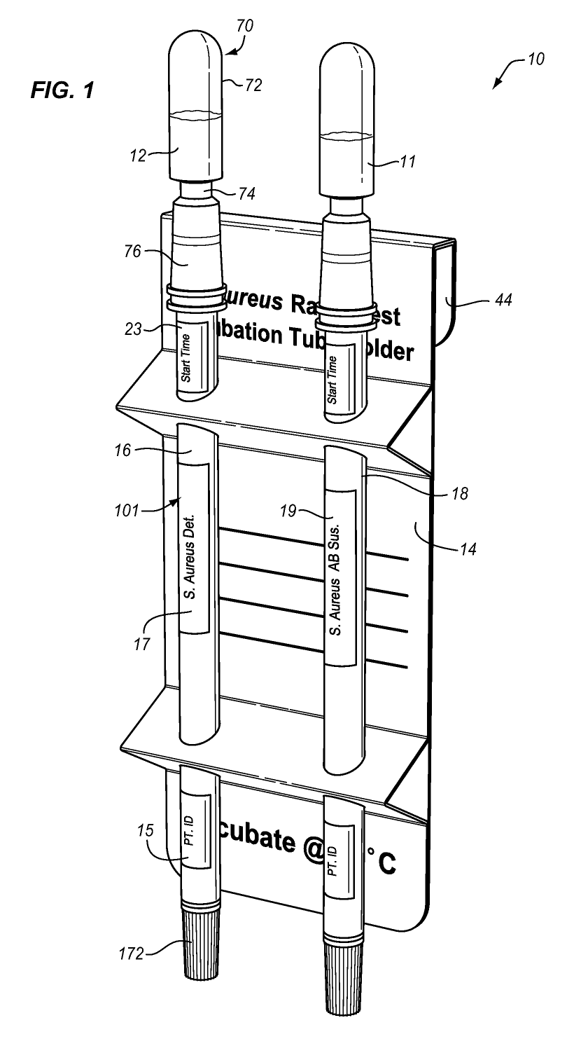

FIG. 1 is a perspective view illustrating a preferred embodiment of a bacteria identification and antibiotic susceptibility/resistance dual collection, incubation, and applicator system 10 according to the invention. Dual system 10 includes a dual holder 14; a bacteria identification collector, incubator, and applicator unit 16; and an antibiotic susceptibility collector, incubator, and applicator unit 18. For ease of reference, we shall refer to the collector, incubator, and applicator units 16 and 18 herein as “incubator units” or “units”, though it should be understood that they perform all three functions. Each of units 16 and 18 include a bulb assembly 70, an incubator body member 101, which preferably is in the form of a tube 101, and an applicator cap 172. Bulb assembly 70 includes a flexible bulb 72, a neck 74, and a connector 76 for connecting the bulb assembly to the incubator body member 101. Each of the bulbs 72 contains a fluid 11 and 12, which will be described in detail below. Each unit 16 and 18 includes labeling 17 and 19, respectively, indicating the test; labeling, such as 15 on which the patient 1D may be written; and labeling, such as 23, on which the start time may be written. Preferably, except for the fluids 11, 12 and labeling 17, 19, both of units 16 and 18 are identical. We shall discuss the detailed structure of units 16 and 18 below.

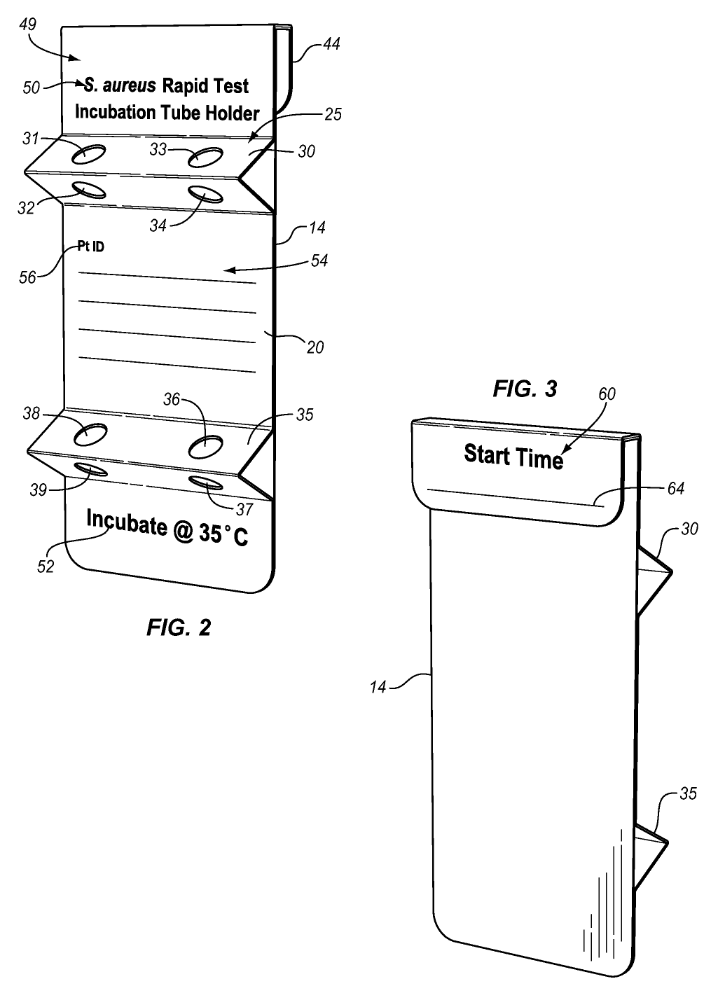

FIG. 2 is a front perspective view of the dual system holder of FIG. 1, and FIG. 3 is a back perspective view of the dual system holder of FIG. 1. Holder 20 includes a holder body member 20 and incubator unit attachment assembly 25 which preferably includes brackets 30 and 35. Bracket 30 includes openings 31, 32, 33, and 34, while bracket 35 includes openings 36, 37, 38, and 39, all of which are sized so that tubes 101 snuggly fit in them. The openings and the structure and material of brackets 30 and 35 are such that brackets 30 and 35 securely hold units 16 and 18 together but allow them to be removed. Preferably, holder 20 is made of cardboard, but may be made of other suitable materials. Holder 14 also includes labeling 49, which includes test identification labeling 50, patient labeling 56, with space 54 for writing patient identification information, incubation directions 52, and timing information 60 (FIG. 3), such as the test start time. Holder 20 also includes a flange 44 which permits the holder to be hung or otherwise attached to a holder support (not shown) such as a clipboard.

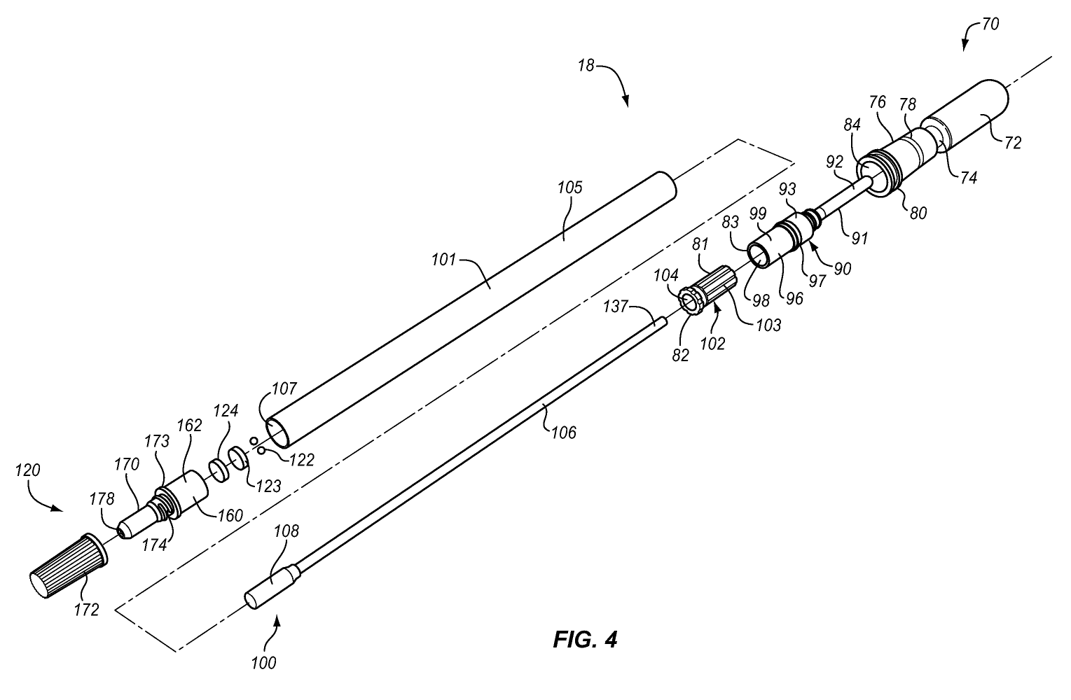

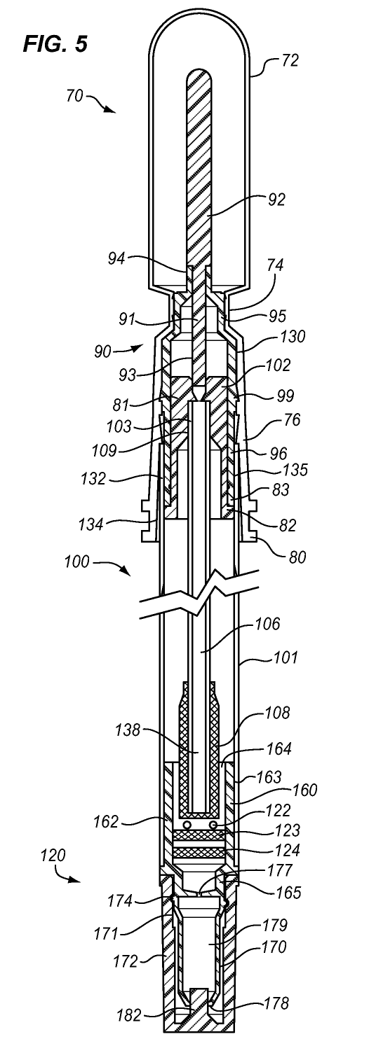

FIG. 4 is an exploded view of one of the collection, incubation, and applicator units 16, 18 of FIG. 1; and FIG. 5 is a longitudinal cross-sectional view of the same. There are four principle subassemblies of each unit 16, 18: bulb assembly 70, breakable stem assembly 90, collector assembly 100, and incubator and applicator assembly 120. Bulb assembly 70 comprises a flexible bulb 72, a neck 74, and bulb connector 76. Connector 76 preferably has a bore 84 having an internal diameter such that body member 101 fits snuggly in it. It also includes internal ribs 78 which provide a location member for breakable stem assembly 90 and external ribs 80 which strengthen the connector and prevent flaring of the end of the connector. Breakable stem assembly 90 includes stem 91 and stem locator/connector 96. Stem 91 comprises upper stem portion 92, preferably within bulb 72, and lower stem portion 93. Connector/locator 96 includes collar 94, neck 95, and connector body member 99 having locator rib 97 which fits between ribs 78 on bulb connector 76 and prevents longitudinal movement of breakable stem assembly 90 with respect to bulb assembly 70. Collar 94 fits snuggly around stem 91 at the juncture of the upper portion 92 and lower portion 93, and stem assembly neck 95 fits snuggly within neck 74 of bulb assembly 70, the upper part 130 of stem locator/connector 96. The upper end 132 of body member 101 fits between the inner surface 134 of bulb connector 76 and the outer surface 135 of stem assembly connector body member 99.

Collector assembly 100 includes collector connector 102, collector rod 106, and collector 108, which in this embodiment is a fabric swab 108. Collector connector 102 has a foot 82 having a larger diameter than the body 81 of the connector and has channels 103 running its length, including through the foot 82. Collector connector 102 fits snuggly into the inner bore 98 of breakable stem connector body member 99, with the foot 82 of collector connector 102 abutting distal end 83 of breakable stem body member 99; and the proximal end 137 of collector rod 106 fits snuggly into inner bore 109 of collector connector 102. Swab 108 is connected, preferably with glue, to the distal end 138 of collector rod 106. Incubator and applicator assembly 120 may include applicator body member 160, reagent beads 122, reagent disc 123, filter 124, and cap 172. Preferably, the beads or disc are made of an absorbent material such as paper or a Pall membrane. In one of the preferred embodiments, a paper disc is used, preferably Alstrom #237 paper. Filter 124 is preferably a Pall BTS membrane. Applicator body member 160 includes a connector portion 162, an applicator tip portion 170, and a tapered intermediate portion 165 connecting the two. Connector portion 162 has an inner bore 164, large enough to snuggly receive optional filter 124 and loosely receive swab 108, and an outer surface 163 that snuggly fits into the bore 107 of incubator unit body member 101. Intermediate portion 165 narrows internally to a small diameter opening 177 and has external threads 174 for connecting to cap 172. Applicator tip 170 has an internal passage 179 that narrows to applicator opening 178. Cap 172 has an internal surface 171 that fits snuggly on threads 174 and an internal pin 182 that firmly plugs applicator opening 178 when cap 172 is placed over the applicator tip 170. Opening 177 is small enough that fluid 11, 12 will not flow through it under the force of gravity due to surface tension, but fluid will flow if the fluid is pressurized by squeezing on bulb 72. This provides a controlled flow of fluid 11, 12 out of opening 178.

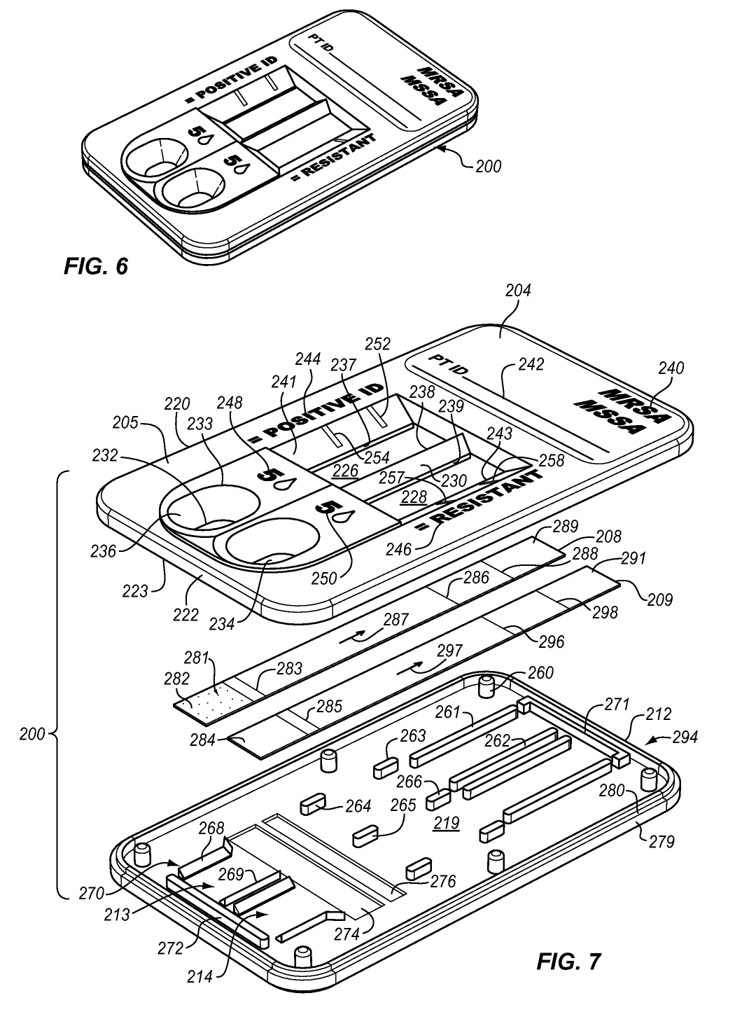

FIG. 6 is a top perspective view of a preferred embodiment of bacteria identification antibiotic susceptibility/resistance dual lateral flow strip assembly 200 according to the invention, and FIG. 7 is an exploded view of the dual lateral flow strip assembly of FIG. 6. For brevity, we shall refer to assembly 200 as the dual flow strip assembly 200, or simply assembly 200. Assembly 200 includes a cover 204, a base 206, and lateral flow strips 208 and 209. Cover 204 preferably comprises a rectangular plate 205 with a downward extending rim 222, though it may have other suitable shapes. Cover 204 has two sample wells 233 and 234 and two windows 226 and 228. Each well 233, 234 has sides, such as 236, that slope inward to an opening, such as 232. Divider 230 separates the windows. The sides, such as 241, of cover 204 adjacent windows 226, 228 and divider 230 slope inward to enable clear viewing of strips 208 and 209. Cover 204 also includes labeling 240 that identifies the test viewable through window 228 as an MRSA/MSSA test, labeling 244 and 246 that identifies the test viewable through windows 226 and 228, labeling 242 that provides for the user entering patient identification information, and labeling 248 and 250 that remind the user that five drops of fluid from applicator tips 178 (FIG. 4) should be placed in the sample wells. It also includes labeling 252, 254, 257, and 258 which indicate where lines should be visible on flow strips 208 and 209 if the tests are positive. That is, if the bacteria is identified as the bacteria to which the bacteriophage of the test is specific, a line will appear on flow strip 208 adjacent line 254; if the assay is functioning properly, a control line appears adjacent line 252; and if the bacteria is susceptible, a line appears on strip 209 adjacent line 258.

Test strips 208 are made of a porous mesh or membrane, preferably nitrocellulous membrane available from many sources, such as Millipore Corporation, 290 Concord Road, Billerica, Mass. A sample pad 282 is in contact with strip 208. In some embodiments, sample pad 282 is a portion of the same membrane from which the strip is made, but preferably it is a separate piece of absorbent material attached to the membrane. In the preferred embodiment, a conjugate 281 specific to the bacteriophage of the test is embedded in the sample pad. This may be done by suspending the conjugate in a fluid and applying the fluid to the sample pad, preferably using a bath into which the pad is dipped, and then drying the pad. Alternatively, the conjugate may in a conjugate area 283 located in the flow strip 208. A conjugate is anything that can bind with the bacteriophage to assist in the identification assay. Well-known conjugates include antibodies; antibodies conjugated to a marker, such as a colored bead; an enzyme; a colloidal particle, such as gold; and biotin, which, when attached to a bacteriophage, can permit the bacteriophage to become attached to a streptavidin-coated object. In the preferred embodiment, the conjugate is a gold conjugated Rabbit bacteriophage antibody. Strip 208 also includes an identification area 286 and a control area 288. Identification area 286 preferably comprises a substance that will fix the bacteriophage of the specific test, which substance is embedded in flow strip 208. Identification area 286 is most preferably a line of antibodies specific to the identification bacteriophage embedded in the strip 208. For example, if the bacteriophage is MP112 (i.e., bacteriophage 112 of MicroPhage™ Incorporated), then the antibody in line 286 is an antibody to the MP112 bacteriophage. Control area 288 preferably comprises a substance that will fix a component of the conjugate that joins the flow at conjugate area 283. Most preferably, it is a line of antibodies to the Rabbit antibody that is in the conjugate area.

Sample pad 284 and lateral flow strip 209 are similar to sample pad 282 and strip 208, and most preferably identical to pad 282 and strip 208. This simplifies the system. However, in some embodiments it may have different constituents. Preferably, strip 209 includes a bacteriophage fixation area 296, and a control area 298, and optionally a conjugate area 285, all preferably comprising the same materials as for strip 208, though the invention contemplates that the materials may be different.

Base 206 is designed to mate with and attach to cover 204 while securely holding strips 208 and 209 in strip beds 213 and 214, respectively. Preferably, base 206 is a rectangular plate 219 having a raised rim 212 about the circumference of the base. Rim 212 is inset a small distance from the edge 279 of place 219 creating a ledge 280. The rim 212 of base 206 snuggly fits within rim 222 of cover 204, and the lower edge 223 of cover rim 222 mates with ledge 280. Posts 260 are formed on base 206 and friction fit into corresponding bores in raised studs in cover 204. Raised studs 261, 262, 263, 264, 265, 266, 268, 269, 271, and 272 form a framework 270 that defines bed 213 for strip pad 282 and 208 and into which strip pad 282 and 208 fit loosely but without play. A similar framework 294 defines bed 214 for pad 284 and strip 209 and in which pad 282 and strip 209 fit loosely without play. Wells 274 and 276 provide reservoirs for excess fluid in case a user applies too much fluid containing the sample.

The bacteria identification and antibiotic susceptibility/resistance system according to the invention is operated as follows. The swab end 108 of collector assembly 100 of unit 16 is used to swab a potential source of a specific bacteria, which in the preferred embodiment may be a nostril of a patient admitted into a hospital. Preferably, the same nostril is also swabbed with a second swab of a second collection incubation and applicator system 18. The swabs are inserted into their respective incubator tube 101. The breakable stem 91 of each unit is broken by holding the lower portion of each of the units 16, 18, preferably by grasping stem connector 76 through bulb connector 96, and bending upper stem portion 92 while squeezing bulb 72 until the stem 91 breaks at collar 94. This forces incubation fluid 11, 12 through channels 103 (FIG. 4) into the respective incubator tube 101. If, in the particular test, reagent beads such as 122 and/or reagent discs such as 123 are used to add one or more reagents, such as an antibiotic, to the incubator fluid 11, 12, then this reagent or reagents are dissolved in the fluid when it reaches the beads or discs. The sample is permitted to incubate at the preferred temperature of 35° C. for a predetermined time, preferably four hours, though the time will vary depending on the bacteria to be identified and the bacteriophage used to identify it. Cap 172 of unit 16 then is removed, and the incubated sample in unit 16 is applied to sample pad 282 of strip 208 via well 233; and cap 172 of unit 18 is removed, and the incubated sample in unit 18 is applied to sample pad 284 of strip 209 via well 234. If the test is such that it is expected that the sample would contain detritus, red blood cells, or other material that could interfere with the flow of the fluid in the pads 282 and 284 and flow strips 208 and 209, then a filter 124 may have been included in the incubator/applicator assembly 120. In this case, as the sample is applied, the detritus, red blood cells, or other material is filtered out. If target bacteria are present, bacteriophage in the incubation fluid 12 will have amplified. The conjugate 281, which is specific to the selected bacteriophage, will attach to the conjugate 281 in the sample pad, and the bacteriophage-conjugate will flow into the strip 208 and then down the strip 208 toward end 289, i.e., in the direction of arrow 287. Alternatively, in the case where the conjugate is in a conjugate area 283 in strip 208, as the bacteriophage flow through conjugate area 283, the conjugate will attach to them. At the fixation area 286, the bacteriophage will attach to the fixation substance, which is specific to the bacteriophage or conjugate, and a visible line of the conjugate, such as colloidal gold or a colored bead, will be created. If the target bacterium is not present in the sample, the bacteriophage will not have amplified, and there will not be sufficient bacteriophage/conjugate complexes to produce a strong visible line. Whether or not bacteriophage are present, conjugate will flow down the strip in the direction 287 and will form a visible line at control area 288, showing that the test is working. In the test strip 209, if the bacterium is susceptible to the antibiotic in the incubation fluid 11, the bacteria will be killed, the bacteriophage will not be amplified, and no line will appear at 257. If the bacterium is resistant to the bacteriophage, it will not be killed, the bacteriophage will be amplified, and a line will appear at 257. In both cases, if the system is working, a control line will appear at 258.

It should be understood that the dual flow strip assembly 200 of FIGS. 6 and 7 can be replaced by two single flow strip assemblies. In fact, in many of the clinical trial tests planned, a pair of single flow strip assemblies is used, with one being used for the identification test, and the other being used for the antibiotic susceptibility/resistance test. Further, both the identification test and the antibiotic susceptibility/resistance test can be performed with a single test unit 19, which includes the antibiotic to be tested, and a single flow strip, such as 208, in a suitable single flow strip holder. However, in such a single unit test, a negative test is ambiguous in that it can indicate either that the bacterium is susceptible or that no bacteria is present. Still, this test is highly useful because it tells the physician, hospital, or other party very quickly and efficiently that an MRSA is present, which is a very dangerous situation in a hospital, calling for immediate isolation and for which rapid, efficient detection is highly desirable. Since it can be done at approximately half the cost of the dual test, such a “single tube” test can be used more broadly, for example, in admitting patients into a hospital. Incubation time for an MRSA screening test is preferably eight to twenty-four hours.

Sometimes a bacterium may be partially resistant or partially susceptible to an antibiotic. In such cases, a line may appear adjacent labeling 257, though it will not be as pronounced as it would be if the bacterium is completely resistant. In such cases, line densities above a certain predetermined density may be deemed to be resistant, and line densities below this density may be deemed to be susceptible. Generally, if no line appears, the amount of bacteriophage present is below the amount associated with the predetermined indicator, i.e., the line density deemed to indicate resistance, and the bacterium is deemed susceptible. If a strong line appears, then the amount of bacteriophage present is above the amount associated with the predetermined indicator, and the bacterium is deemed resistant.

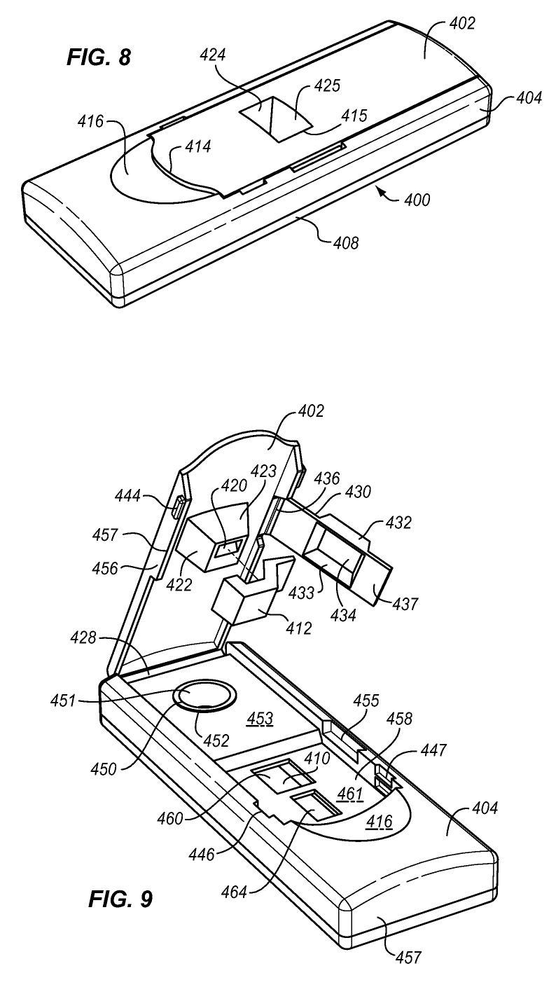

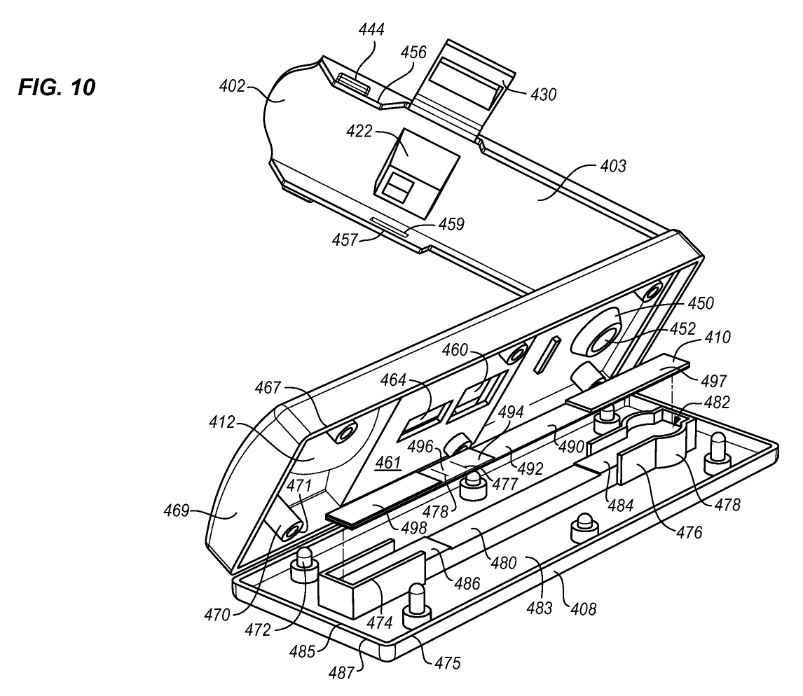

FIG. 8 is a top perspective view of an alternative embodiment of a bacteria identification assembly 400 according to the invention, FIG. 9 is a top perspective view of the bacteria identification assembly of FIG. 8 with the top cover 402 opened, and FIG. 10 is a bottom perspective view of the bacteria identification assembly of FIG. 8 with the base 408 open. Identification assembly 400 includes a cover 402, a body member 404, a base member 408, a sample pad and flow strip assembly 410, and a membrane 412. Preferably, membrane 412 is a blood barrier membrane, such as BTS membrane available from Pall Corporation, 2200 Northern Boulevard East Hills, N.Y. 11548. Cover 402 is preferably a plate 403 with flanges and other molded parts as described below, and base 408 is preferably a rectangular plate 483 with an upturned rim 487. Base member 404 preferably is roughly a rectangular box 469 with an open bottom and other features as described below. Cover 402 comprises a sample port 415 having a top opening 424, a funnel 422 having sloping sides 423, 424, and 425 and a bottom opening 420, and a filter cover 432. The forward side 423 preferably has a steeper, most preferably, vertical slope; the sides slope less to move the sample toward bottom opening 420, while the back side 425 has a lesser slope to give the sample some inertia to move along the flow strip 410. Body member 404 includes an indented portion 416, which, in combination with lip 414 on cover 402. allows a fingertip to be inserted into indent 416 and under lip 414 to flip cover 402 open. Cover 402 also includes hinge 428, flanges 456, and tabs 444 on both sides. Flanges 456 extend from cover 402 into well 458 in body member 404 with tabs 444 mating with slots 446 and then snapping under lips 447 to hold cover 402 in the closed position. Indent 455 in body member 404 receives the bulge created by hinge 436 when cover 402 is closed. Cover 402 also includes filter flap 430. Flap 430 includes a funnel 432 with sloping sides 433 which slope to flap sample port 434. Filter 412 is shaped to fit snuggly over funnel 422, and funnel 432 is shaped to fit snugly over filter 412. Flap 430 is attached to cover 402 via hinge 436. Distal end 437 of flap 430 snaps under a lip 457 on cover 402 into a groove 459 (FIG. 10) to hold filter 412 in place.

Sample pad and flow strip assembly 410 is preferably a porous mesh material, such as nitrocellulous membrane available from many sources, such as Millipore Corporation, 290 Concord Road, Billerica, Mass. It includes a conjugate area 492 which contains a conjugate embedded in the porous material and a reading area 496 which preferably includes an identification area 477 and a control area 478 as described above. In the preferred embodiment, the conjugate is a gold colloidal conjugate. The sample port 415 directs the sample through filter 412 to strip 410 at sample area 494 between conjugate area 492 and reading area 496. The ends of strip 410 are preferably thicker than the flow region 490 which includes conjugate area 492, sample area 494, and reading area 496. This thicker portion forms absorption pads 497 and 498. Body member 404 includes sample port 460, reading window 464, and chase buffer port 450. Base 408 includes framework 474 and 476 which forms a bed 482 for strip 410. The ends 486 and 484 of bed 482 are raised, while the middle portion 480 of bed 482 is indented a small amount. This structure, together with the thickness difference between the ends 497, 498 and flow region 490 of strip 410, allows fluid to easily flow laterally in the flow portion 490. Studs 472 formed in base 408 fit into bores 471 of posts 470 in body member 404 with a tight friction fit to securely hold body member 404 to base member 408, with the bottom end 467 of body member 404 abutting the top 485 of rim 487 and strip 410 trapped between the bottom side of well 461 and the bed 482.

The bacteria identification system 400 operates as follows. An incubated sample from unit 16 is applied to sample area 494 of strip 410 via sample port 415. After a minute or so to allow the sample area to saturate, a finger is inserted under lip 414, cover 402 is lifted, and a chase buffer, preferably comprising TBS (tris buffer saline) containing a small amount of Tween 20 surfactant, preferably about 0.05%, is applied. The chase buffer washes the conjugate into the sample area, where it conjugates with the bacteriophage in the sample area 494. The conjugate/bacteriophage complex continues to flow downstream to the identification area 477 where the bacteriophage, if present, is fixed, and then to the control area 478, where the conjugate is fixed by a fixation substance, such as an antibody. If the target bacteria was present in the sample, the bacteriophage will have amplified and a colored line will be present in the identification area, which color can be read through window 464. System 400 can also be used as a susceptibility/resistance test system using the susceptibility/resistance test teachings associated with unit 18 and flow strip 209 above.

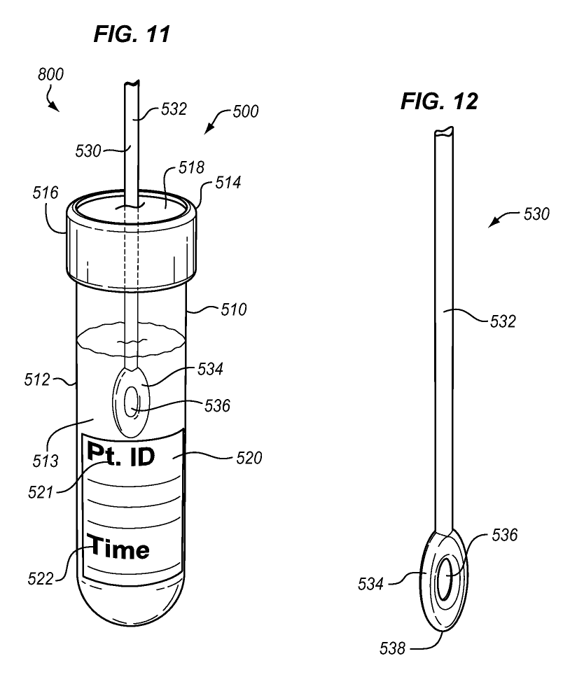

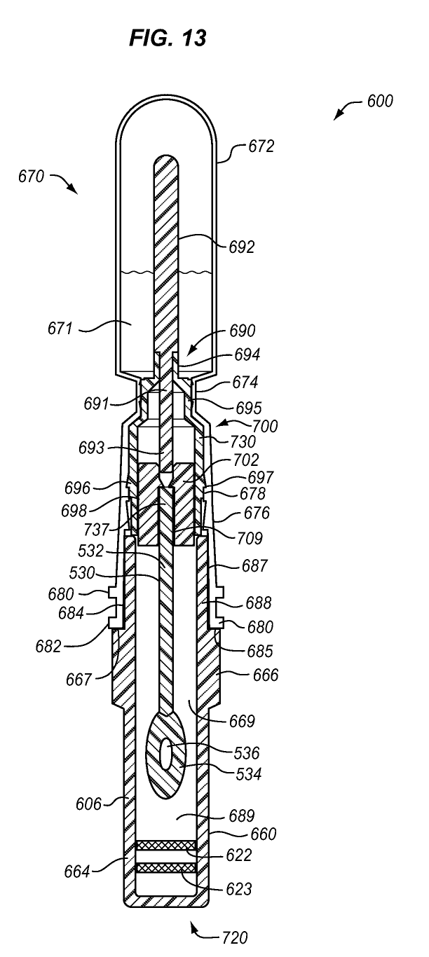

FIG. 11 is a perspective view of a preferred embodiment of an alternative embodiment of a sample collection system 500 according to the invention. Collection system 500 includes collector assembly 530 and blood vial 510. FIG. 12 is a perspective view of the sample collection unit 530 of FIG. 11, and FIG. 13 is a cross-sectional view of an alternative embodiment of a collector-incubator-applicator unit 600 according to the invention, including the collection unit of FIG. 12. Just the collector assembly 530 of the collector-incubator-applicator unit 600 is shown in FIG. 12.

Blood vial 510 includes a container 512 having a cap 514 and a label 520 for entering patent identification information 521 and other information, such as time 522. Cap 514 includes a vial attachment member 516 and a resilient diaphragm 518. Attachment member 516 is preferably a threaded ring-shaped adapter, preferably made of plastic, metal, or other suitable material. It may also be a snap-on attachment member. In use, container 512 will contain a blood sample 513. Collector assembly 530 preferably includes a shaft 532 and an enlarged collection head 534 having an eye 536. Collection head 534 preferably has a thin tip 538, which preferably is sharp enough to easily pierce flexible insert 518 but not so sharp as to create a hazard of cutting a finger or glove. Resilient diaphragm 518 is preferably made of rubber or other material that may be pierced by head 534 but seals upon withdrawal of the head 534.

Collector-incubator-applicator unit 600 comprises bulb assembly 670, breakable stem assembly 690, collector assembly 700, and incubator-applicator assembly 720. Incubator-applicator assembly 720 comprises incubator 606 which also forms applicator cap 664. Bulb assembly 670 comprises a flexible bulb 672, a neck 674, and bulb connector 676. Bulb connector 676 is preferably a roughly tubular structure having a bore 684 having an internal diameter such that end 684 of applicator cap 664 fits snuggly in it. It also includes internal ribs 678, which provide a location member for breakable stem assembly 690, and external ribs 680, which strengthen the connector and prevent flaring of the distal end 682 of the connector. Breakable stem assembly 690 includes stem 691 and stem locator/connector 696. Stem 691 comprises upper stem portion 692, preferably within bulb 672, and lower stem portion 693. Connector/locator 696 includes collar 694, neck 695, and connector body member 699 having locator ribs 697 which straddle ribs 678 on bulb connector 676 and prevents longitudinal movement of breakable stem assembly 690 with respect to bulb assembly 670. Collar 694 fits snuggly around stem 691 at the juncture of the upper portion 692 and lower portion 693; and stem assembly neck 695 fits snuggly within neck 674 of bulb assembly 670, and is preferably integrally formed with the upper part 730 of stem locator/connector 696. Collector assembly 700 includes collector connector 702, collector shaft 532, and collector head 534, which were discussed above. Collector connector 702 fits snuggly into the inner bore 698 of breakable stem connector body member 699, and includes channels similar to the channels 103 of collector connector 102 to allow fluid to pass from the bulb 672 to incubator cap 664 when the breakable stem 691 is broken. Proximal end 737 of collector shaft 532 fits snuggly into inner bore 709 of collector connector 702. Incubator and applicator cap 664 has a proximal end 660, an intermediate portion 666, and a distal end 688. Distal end 688 has an outside surface 687 with a diameter that fits snuggly within inner bore 684 of bulb connector 676. Intermediate portion 666 of applicator cap 664 preferably has an external diameter that is wider than the external diameters of the proximal and distal ends, which permits distal end surface 667 of intermediate portion 666 to abut the end surface 685 of bulb connector 676 when cap distal end 688 is pushed into bulb connector 676. Applicator cap 664 has an internal cavity 669, preferably of cylindrical shape, of an inner diameter such that collector head 534 fits within it with room for broth fluid to flow around the head 534. The internal volume of cavity 669 is preferably such that, when the collector-incubator-applicator unit 600 is assembled and held vertically, the volume of broth in bulb 672 fills the end 689 that holds collector head 534 with the eye 536 of head 534 fully immersed in the broth. In some embodiments, the volume of end 689 will also include one or more porous reagent discs 622 and 623 that contain reagents that assist in developing bacteriophage that enhance the test, retard the development of bacteriophage that interfere with the test, or contain some other reagent that enhances the test. These reagents have been discussed above.

The sample collection system 500, together with the collector-incubator-applicator unit 600, forms a bacteria identification system 800 according to the invention, which preferably is used to identify bacteremia. Bacterial identification system 800 operates as follows. The head 534 of collector assembly 530 is inserted through the resilient diaphragm 518 of blood culture vial 510, allowing a drop of blood 513 to enter eye 536. The collection head 534 is withdrawn from vial 510, inserted into applicator cap 664, and breakable stem 691 is broken while bulb 672 is being squeezed as discussed above in connection with FIG. 5, which forces the incubation fluid 671 in the bulb to enter applicator cap 664, preferably filling it to a level higher than eye 536. The sample is permitted to incubate for a predetermined period of time, preferably about four hours for a blood bacteremia test, and the sample is applied to either flow strip sample pad 282 via port 233, flow strip sample pad 284 via port 234, or flow strip sample area 496 via port 415, depending on whether a bacteria identification test or antibiotic susceptibility/resistance test is being performed and which identification assembly is used. The test then proceeds as discussed above.

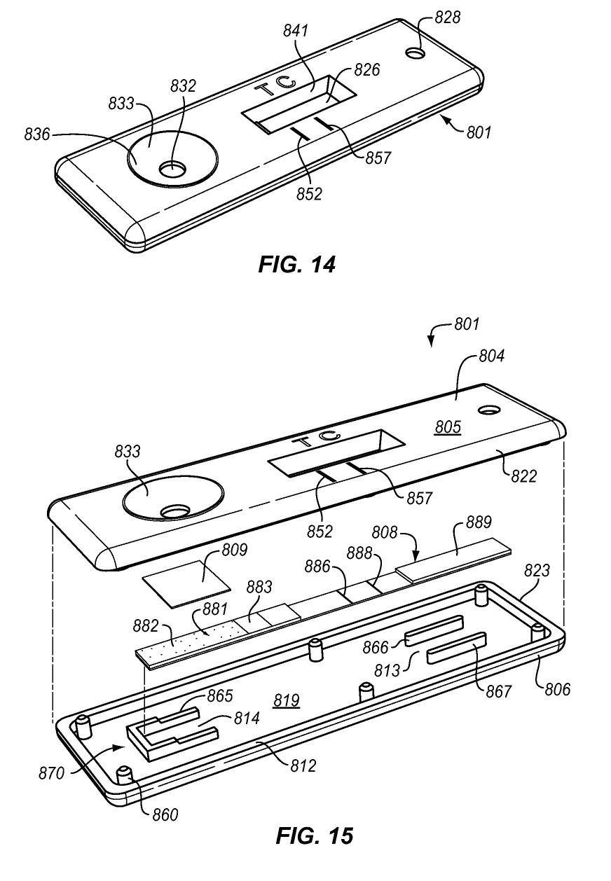

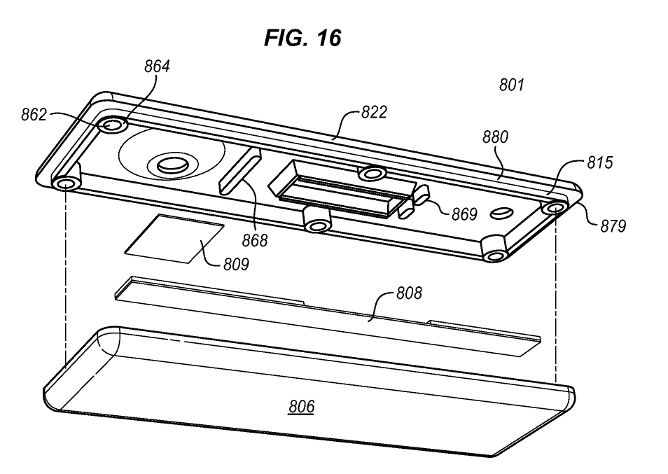

FIG. 14 is a top perspective view of another preferred embodiment of bacteria identification antibiotic susceptibility/resistance lateral flow strip assembly 801 according to the invention, FIG. 15 is a top exploded view of the lateral flow strip assembly of FIG. 14, and FIG. 16 is a bottom exploded view of the lateral flow strip assembly of FIG. 14. We shall refer to assembly 801 as the simplified flow strip assembly 801, or simply assembly 801. Assembly 801 includes a cover 804, a base 806, a red blood cell filter 809, and lateral flow strip 808. Cover 804 preferably comprises a rectangular plate 805 with a downward extending outer rim 822, though it may have other suitable shapes. As shown in FIG. 16, cover 804 also includes a downward extending flange 815 that is set back from the outer edge 879 to from a ledge 880. Cover 804 includes sample well 833, window 826, and vent 828. Well 233 has side 836 that slopes inward to opening 832. The sides, such as 841, of cover 804 adjacent window 826 slope inward to enable clear viewing of strip 808. Cover 804 also may include labeling as described in connection with FIG. 7 above. It also includes labeling 852, 857 which indicate where lines should be visible on flow strip 808 if the test is positive. That is, if the bacteria is identified as the bacteria to which the bacteriophage of the test is specific, a line will appear on flow strip 208 adjacent line 852; if the assay is functioning properly, a control line appears adjacent line 857.

Sample pad 882 and flow strip 808 are made of a porous mesh or membrane as described above. Sample pad 882 preferably comprises a porous membrane that lies on top of and in contact with strip 808. In the preferred embodiment, a conjugate 881 specific to the bacteriophage of the test is embedded in the sample pad. Alternatively, the conjugate may be embedded in a conjugate area 883 in strip 808. Strip 808 also includes an identification area 886 and a control area 888. Identification area 886 preferably comprises a substance that will fix the bacteriophage of the specific test, which substance is embedded in flow strip 808. Identification area 886 is most preferably a line of antibodies specific to the identification bacteriophage embedded in the strip 808, as described above. Control area 888 preferably comprises a substance that will fix a component of the conjugate that joins the flow at conjugate area 883. Most preferably, it is a line of antibodies to the Rabbit antibody that is in the conjugate area. Filter 809 is preferably a Pall BTS membrane. Absorption pad 889 preferably is formed by a thicker part of the strip.

Base 806 is designed to mate with and attach to cover 804 while securely holding strip 808 in strip beds 813 and 814. Preferably, base 806 is a rectangular plate 819 having a raised rim 812 about the circumference of the base. The rim 812 of base 806 snuggly fits within rim 815 of cover 804, and the ledge 880 of cover 805 mates with the upper surface 823 of rim 812. Posts 860 are formed on base 806 and friction fit into corresponding bores 862 in raised studs 864 in cover 804. Raised studs 865, 866, and 867 form a framework 870 that defines beds 813, 814 for strip 808 and into which strip 808 fits loosely but without play. Flanges 868 and 869 on the bottom of cover 804 also assist in holding strip 808 in place.

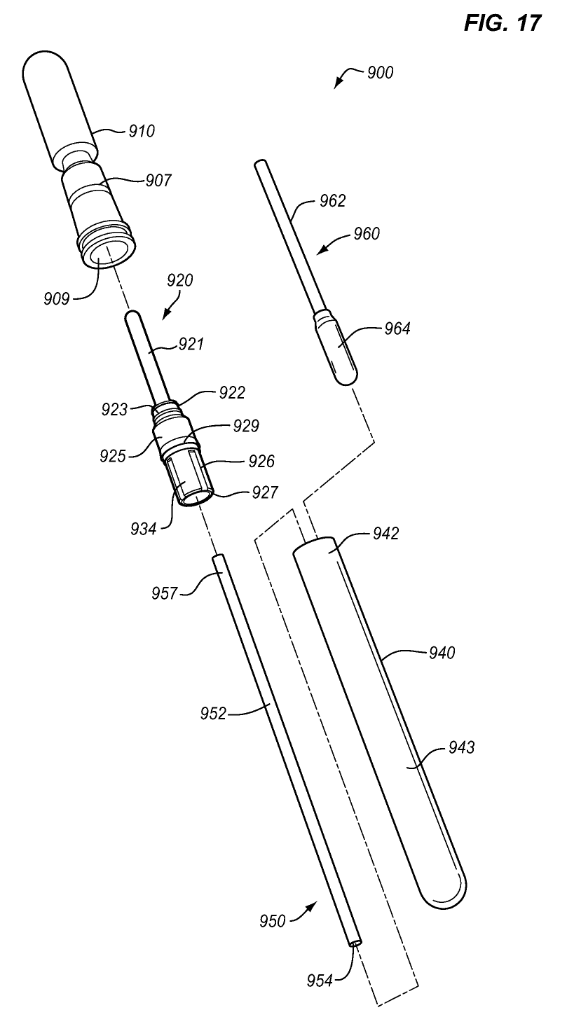

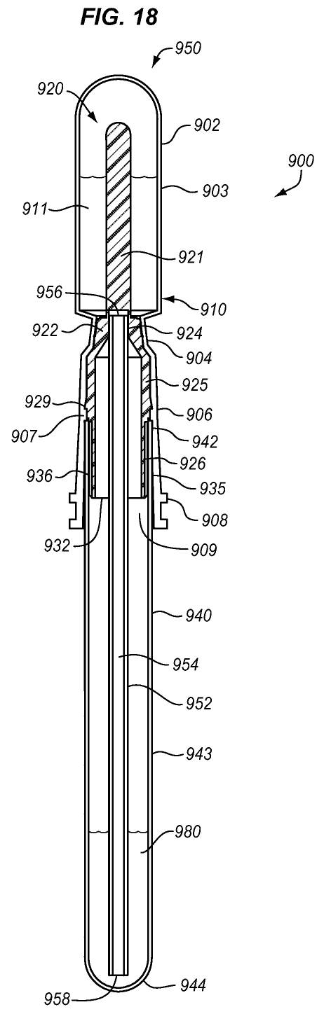

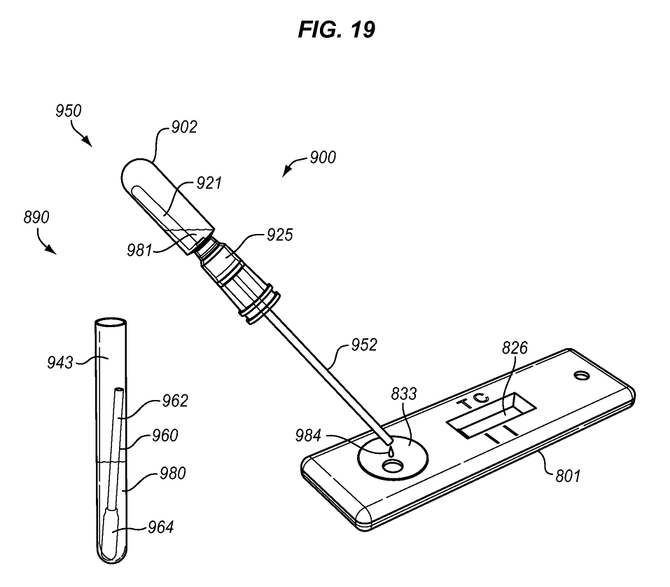

FIG. 17 is an exploded view of another preferred embodiment of a collection, incubation, and applicator system 900 according to the invention; FIG. 18 is a cross-sectional view of the incubation and applicator system portion 980 of the system of FIG. 17; and FIG. 19 illustrates a preferred embodiment of the collector, incubation, and applicator system 900 according to the invention, showing how the collector 960 is inserted into the incubator 940 and the use of the applicator 950. There are five principle subassemblies of each unit 900: bulb assembly 910, breakable stem assembly 920, collector assembly 960, incubator assembly 940, and applicator assembly 950. Applicator assembly 950 includes bulb assembly 910 and transfer tube 952. Bulb assembly 910 comprises a flexible bulb 902, a neck 904, and bulb connector 906. Connector 906 preferably has a bore 909 having an internal diameter such that body member end 942 of incubation container 943 fits snuggly in it. It also includes internal ribs 907, which provide a location member for breakable stem assembly 920, and external ribs 908, which strengthen the connector and prevent flaring of the end of the connector 906. Breakable stem assembly 920 includes stem 921 and stem locator/connector 925. Connector/locator 925 includes neck 922 with friction rings 923 and connector body member 934 having locator rib 929 which snaps behind rib 907 on bulb connector 925 and prevents longitudinal movement of breakable stem assembly 920 with respect to bulb assembly 910. Bulb assembly neck 904 fits snuggly around stem neck 922, and stem 921 is breakably connected to neck 922. The distal end 942 incubator container 943 fits between the inner surface 935 of bulb connector 906 and the outer surface 936 of stem assembly connector locator/connector 925. Grooves 926 in the end 927 of stem assembly connect/locator 925 provide increased flexibility to allow the end 942 of incubator container 943 to slip more easily over the end 927 of stem assembly 920. Liquid transfer tube 952 is preferably a hollow cylinder having an internal channel 954. The proximal end 957 of tube 952 fits snuggly into a bore 924 in neck 922 of stem assembly 920.

Collector assembly 960 includes collector handle 962 and collector 964 which, in this embodiment, is a fabric swab 924. The handle 962 may be a hollow tube made of the same material as tube 952.

The operation of the lateral flow strip assembly 801 as shown in FIGS. 14-16 and the collection, incubation, and applicator system 900 as shown in FIGS. 17 and 18 will be discussed in connection with FIG. 19. Together these form a bacteria identification system 890. A source of bacteria, such a person's nose, is swabbed with swab 964 holding collector 960 by handle 962, and the collector is dropped into incubator container 943. Incubator container 943 is pushed into the bore 909 of bulb connector 906 until it if firmly connected as shown in FIG. 18. Then the outside 903 of bulb 902 is firmly grasped and bent so that breakable stem 921 breaks where it connects to neck 922. Fluid 911 (FIG. 18) can then flow under gravity and the compression of bulb 902 into incubator container 943 until it fills end 944. The bulb 902 may be squeezed several times to thoroughly mix the liquid and the sample in the swab 964. After a suitable incubation period, the bulb is again squeezed and released to suck up some of incubated liquid 980, shown as fluid 981 in bulb 902 in FIG. 19. The applicator assembly 950 and incubator container 943 are then separated and the applicator is used to apply a suitable amount of incubated sample solution 984 to a sample pad and flow strip assembly sample well, such as 833. The sample flows through opening 832 to sample pad 882, with red blood cells being filtered out in filter 809 (FIG. 15). If there is phage in the sample specific to the conjugate, the conjugate 881 attaches to the phage. Alternatively, the sample flows through conjugate area 883, where the phage picks up the conjugate. In either case, the phage-conjugate complex proceeds to the identification area 886 and control area 888. As described above, a detectable line appears adjacent label 852 if the test is positive, and a detectable control line appears adjacent label 857 to show the test is valid. The remainder of the sample finally flows to adsorption pad 889.

The collector/incubator/applicator and flow strip assembly parts, except for the swabs and strip membranes, the materials of which were described above, are preferably made of a suitable medical grade plastic. The breakable stem assemblies, such as 90 and 920, are preferably made of a brittle, more rigid plastic, while the bulb assemblies, such as 70 and 910, are made of a more flexible plastic that will not break when bent or twisted. The parts such as collector connectors, such as 102, applicator body member 160, and cap 172 are molded of a harder plastic. The plastic of the bulb, such as 72 and 902, and incubator containers, such as 101 and 943, are preferably clear plastic so that the liquid can be seen through them. The flow strip covers, such as 204 and 805, and bases, such as 206 and 806, are preferably made of a moldable plastic.

The liquids 11, 12, 671, and 911 have been previously described in U.S. patent application Ser. No. 11/933,083 filed Oct. 31, 2007 and PCT Patent Application No. PCT US08/066,962 filed Jun. 15, 2008, which were incorporated by reference above.

The system of the invention provides a broth that includes substances that enhance bacteriophage amplification. Lauric acid, other fatty acids, and their derivatives ameliorate the effects of β-lactam antibiotics on phage amplification in methicillin-resistant S. aureus (MRSA) hosts. This property enhances the performance and utility of bacteriophage-based tests in detecting, classifying, and distinguishing MRSA from methicillin-susceptible S. aureus (MSSA). Other fatty acid compounds that positively stimulate bacteriophage amplification include saturated fatty acids: caproic acid, caprylic acid, capric acid, and myristic acid; conjugated fatty acids: glycerol monolaurate; and unsaturated fatty acids: oleic acid and linoleic acid. For the purposes of this invention, the term “fatty acid” shall refer to all such compounds and related compounds. Pyruvic acid and related compounds such as its salts, particularly sodium pyruvate, have been found to stimulate bacteriophage amplification, leading to better assay performance.

The methods and substances that enhance bacteriophage amplification are preferably used in combination with substances and methods that inhibit replication in potentially cross-reactive, non-target bacteria, and use this inhibition to increase the selectivity of the phage-based diagnostic process. We shall describe three embodiments of the inhibition process herein: 1) inhibiting the growth of potentially cross-reactive bacteria while allowing growth of the target bacteria; 2) selectively removing potential cross-reactive bacteria from a sample using selective binding agents attached to some support (i.e., microparticles); and 3) selectively destroying potentially cross-reactive bacteria. These embodiments are intended to be illustrative, though the invention is not limited to these embodiments. Other methods with the same results can be contemplated by those skilled in the art.

Inhibition of potentially cross-reactive bacteria can be accomplished using substances such as sodium chloride (in high concentration), Polymyxin B, Polymyxin E, other Polymyxins, and metal compounds such as potassium tellurite. These substances inhibit the growth of some coagulase negative Staphylococcus (CNS) while allowing the growth of Staphylococcus aureus. These compounds can also significantly inhibit or retard replication of bacteriophage in CNS while minimally affecting replication in Staphylococcus aureus. The usage of selective media to differentially affect the efficiency and timing of phage replication is a novel method for improving the specificity of bacteriophage-based bacterial diagnostic methods.

The broth formulations according to the invention also inhibit phage attachment and/or replication in potentially cross-reactive, non-target bacteria, and use this inhibition to increase the specificity of the phage-based diagnostic process. The inhibiting may comprise the addition of an inhibiting substance or the use of an inhibiting process. Three embodiments of the inhibition process are described herein: 1) inhibiting the growth of potentially cross-reactive bacteria while allowing growth of the target bacteria; 2) selectively removing or blocking potential cross-reactive bacteria using selective binding agents; and 3) selectively destroying potentially cross-reactive bacteria. These embodiments are intended to be illustrative, though the invention is not limited to these embodiments. Other methods with the same results can be contemplated by those skilled in the art.

Inhibition of potentially cross-reactive bacteria can be accomplished using salts such as sodium chloride (in high concentration), divalent cations, antibiotics such as Polymyxin B or E, antiseptics such as acriflavine, metal compounds such as potassium tellurite, and iron chelators such as desferoxamine. These compounds inhibit the growth of some coagulase negative Staphylococcus (CNS) while allowing the growth of Staphylococcus aureus. These compounds can also significantly inhibit or retard replication of bacteriophage in CNS while minimally affecting replication in Staphylococcus aureus. The usage of selective media to differentially affect the efficiency and timing of phage replication is a novel method for improving the specificity of bacteriophage-based bacterial diagnostic methods.

Removal or blocking of non-target bacteria may be accomplished using antibodies, bacteriophage selective for the non-target bacteria, or other compounds that selectively bind to non-target bacteria. For a Staphylococcus aureus identification test, removal of CNS species can be beneficial. Binding of these compounds to non-target bacteria may be sufficient to block subsequent binding to those bacteria by bacteriophage that are inadequately specific for the target bacteria, thus preventing non-specific infection and replication in non-target bacteria. Alternatively, these compounds may be attached to other substrate such as microparticles, magnetic beads, or solid substrates. When incubated with a sample, potential non-target bacteria will selectively bind to the substrate. The substrate then can be physically removed from the sample. Separation methods include centrifugation of microparticles, application of a magnetic field for isolating magnetic beads, or other separation processes.

Selective destruction of non-target bacteria can be accomplished using antibacterial compounds that selectively destroy non-target bacteria such that they are not susceptible to phage infection while leaving target bacteria largely unharmed and susceptible to phage infection. Such compounds include a) selective antibiotics and b) bacteriophage that selectively bind to and/or infect potentially cross-reactive, non-target bacteria. The latter are complimentary bacteriophage to the primary bacteriophage used to selectively infect the target bacteria in the sample. Complimentary bacteriophage can destroy non-target bacteria by successfully infecting and lysing those non-target bacteria such that phage infection by the primary bacteriophage is eliminated or significantly reduced. Complimentary bacteriophage can also be used to destroy non-target bacteria by a process known as lysis from without. Lysis from without refers to the destruction of a bacterium when hundreds or thousands of phage particles bind to its cell wall. This process can be utilized in this invention by adding a high concentration of complimentary phage to the sample such that large numbers of complimentary phage quickly and selectively bind to potentially cross-reactive bacteria. Under pressure of multiple phage binding, the cross-reactive bacteria can be made to burst, eliminating them as a focus for phage infection by the prime bacteriophage.

Specific examples of incubation broths 11, 12 that are used with the invention are given in the examples below.

### Example 1

A single test unit MRSA screening test was prepared as follows. A basic broth was prepared by adding sodium pyruvate in a concentration of 27 μg/ml to a TSB (tryptic soy broth) base. This basic broth was autoclaved at 121° C. for 55 minutes. The following ingredients then were added: lauric acid to a concentration of 12 μg/ml (micrograms per milliliter); deferoxamine to a concentration of 500 μg/ml; Na cefoxitin to a concentration of 2 μg/ml; and polymyxin E to a concentration of 10 μg/ml. A phage “cocktail” containing three varieties of phage, namely MP112, MP131, and MP506, at a concentration of 1.67×105 pfu/ml for a total phage concentration of 5×105 pfu/ml was added to the broth. A total both volume of 0.75 ml was placed in bulb 72, and the MRSA screening test was performed as above with an incubation time of eight to twenty-four hours, preferably twelve hours. The incubation time is generally somewhat variable as hospital staffs are busy and come on and off duty at various times; therefore, a test needs to have some flexibility. One hundred fourteen patients were tested, under a confidential test protocol, using this MRSA screening test and also tested using a conventional laboratory-based test, the BBL Chromagar test available from Becton Dickinson. Of the one hundred fourteen tests, fifteen correctly tested positive; that is, the positive result agreed with the conventional laboratory test. That is, the test had 100% sensitivity. Of the one hundred fourteen tests, there were ninety-seven that tested negative that agreed with the conventional laboratory tests and two tested positive but were shown to be false positives by the conventional laboratory test. That is, there was better than 97% specificity with the test. This is remarkable since the tests were not done in a laboratory, but simply by a normal hospital nursing staff under the direction of a physician.

### Example 2

An MRSA single unit screening test as described in Example 1 above was prepared, except that the antibiotics were not added to the broth liquid in the bulb 72, but instead were added via discs, such as 124. The discs were paper discs, specifically Alstrom #237, but any absorbent disc or material can be used. The discs are impregnated with antibiotic by dissolving the antibiotics in a solution, preferably 70% methanol, applying the solution to the discs, and drying. The discs provided Na cefoxitin to a concentration of 2.75 μg/ml and polymyxin E to a concentration of 35 μg/ml. The discs may be used when it may be desirable to store the units 16, 18 for a period of time before use. In liquid form, Na cefoxitin is stable only for a few days, and polymyxin for a few weeks. Thus, a disc is used when the units may be stored longer than a few weeks.

### Example 3

Another MRSA single unit test was prepared as described in Example 1, except that the Na cefoxitin was provided in a disc.

### Example 4

An MRSA dual unit test as shown in FIG. 1 with an identification unit 16 separate from the antibiotic susceptibility/resistance unit 18 was prepared. The liquid 12 for the identification unit 16 was prepared as for Example 1 above, except that the lauric acid was at a concentration of 20 μg/ml, the polymyxin E was at a concentration of 30 μg/ml, and, of course, there was no cefoxitin. The liquid 11 for the antibiotic susceptibility/resistance unit 18 was the same, except that there was no deferoxamine or polymyxin E, and the Na cefoxitin liquid was provided to a concentration of 2.0 μg/ml.

### Example 5

An MRSA dual unit test as shown in FIG. 1 with an identification unit 16 separate from the antibiotic susceptibility/resistance unit 18 was prepared. The liquid 12 for the identification unit 16 was prepared as in Example 3. The liquid 11 of the antibiotic susceptibility/resistance unit 18 was prepared as in Example 2, except that the lauric acid concentration was 20 μg/ml, there was no deferoxamine or polymyxin, and the Na cefoxitin concentration was 2.4 μg/ml.

### Example 6

A dual test unit bacteremia screening test for a bacteremia in combination with the BacTec continuous blood culturing instrument made by Becton Dickinson was prepared as follows. A basic broth was prepared by adding sodium pyruvate in a concentration of 27 μg/ml to a TSB base. This basic broth was autoclaved at 121° C. for 55 minutes. For the identification test, the following ingredients then were added: lauric acid to a concentration of 25 μg/ml (micrograms per milliliter) and polymyxin E to a concentration of 55 μg/ml. The broth was autoclaved for fifteen minutes at a temperature of 121° C. A phage “cocktail” containing four varieties of phage, namely MP112, MP87, MP131, and MP506, at a concentration of 8.33×105 pfu/ml, and a fifth bacteriophage, MP115, at a concentration of 1.67×106 pfu/ml for a total phage concentration of 5×106 pfu/ml was added to the broth. A total broth volume 12 of 1.5 mil was placed in bulb 72 of unit 16. The liquid 11 for the antibiotic susceptibility/resistance unit 16 was prepared by adding lauric acid to a concentration of 20 μg/ml and a disc resulting in Na cefoxitin at a concentration of 3.0 μg/ml to the base broth. A phage “cocktail” containing four varieties of phage, namely MP112, MP87, MP131, and MP506, at a concentration of 5.0×105 pfu/ml, and a fifth bacteriophage, MP115, at a concentration of 1.00×106 pfu/ml for a total phage concentration of 3×106 pfu/ml was added to the broth. A total broth volume of 0.75 ml was placed in bulb 72 of unit 18. This formulation has been tested in the laboratory using a pipette to transfer the blood sample from the blood culture vial to the ID test unit and the antibiotic susceptibility unit.

### Example 7

A dual test unit bacteremia screening test for use in combination with the BacT/Alert automated microbial detection system of Biomerieux, Inc., 100 Randolf Street, Durham, N.C. 27712 using the SA/SN formulation for the blood culture fluid was prepared as follows. The identification unit broth was prepared as described in Example 6, except that the concentration of polymyxin E was 150 μg/ml. The antibiotic susceptibility/resistance unit liquid 11 formulation was identical to that of Example 6. This formulation uses more polymyxin E because the BacT/Alert blood culture fluid contains a resin that is believed to interact with the polymyxin E and reduce its effectiveness. Again, pipettes were use to transfer the blood.

### Example 8

A dual test unit bacteremia screening test for use in combination with the BacT/Alert automated microbial detection system using their FA/FN blood culture formulation was prepared as follows. The identification unit broth was prepared as described in Example 6, except that the concentration of polymyxin E was 200 μg/ml. The antibiotic susceptibility/resistance unit liquid 11 formulation was identical to that of Example 6. Here, even more polymyxin E was used for the reasons given in Example 7.

The tests as described in Examples 2 through 5 tested similarly in the laboratory to the samples of Example 1. For the bacteremia tests of Examples 6 through 8, laboratory tests showed 91% sensitivity and 100% selectivity. All of the above examples are currently being clinically tested in confidential tests in actual patients. So far, the unofficial feedback is that the results are similar to those of Example 1.

It is apparent that one skilled in the art, after reading the above, will understand that many other broth formulations may be prepared based on the teachings of the invention.

It is a feature of the invention that the bacterium detection and identification process and the antibiotic susceptibility and resistance determination can be made in a conventional hospital floor, clinic, or physician's office without the need for specially trained laboratory personnel. This not only lowers the cost of the tests but also greatly increases their speed. Those skilled in the art understand that, no matter how fast a test that requires a laboratory is, it always ends up taking a day or more. That is because the sample must be collected on the hospital floor, clinic, or physician's office and then transferred to the laboratory. This is rarely done immediately because hospitals, clinics, and physician's offices and laboratories all have schedules and procedures that must be adhered to for test accuracy and reasons of economy. For example, a hospital cannot have someone on hand to immediately run a sample down to the lab every time one is taken. Rather, samples are collected and at particular hours are assembled and taken to the lab according to secure and documentable procedures. If a patient comes in late in the day and/or the sample is taken at night, it may not be taken down to the laboratory until the next morning. Further, the laboratory does not immediately do the test as soon as the samples arrive. Rather, test runs are usually done at specific times during the day and generally are not all done at once. A test sample may wait many hours or even half a day in the laboratory before the staff can get to it. The result then needs to be recorded and checked. Finally, the process of reporting it back to the hospital floor can also take hours or longer. Being able to have one conventionally trained person, such as a nurse, take the sample, perform the test, and record it on the patient's chart, dramatically shortens the time for the test.

A related feature of the invention is that the collector/incubator/applicator system 10, 16, 18, 600 according to the invention results in accurate tests that can be repeatably performed by conventionally-trained health care professionals. Immediately after it is collected, the sample may be quickly enclosed in a secure, sealed environment, e.g., incubator tube 101. The incubation fluid 11, 12, 671, 911 may then be combined with the sample without breaking the sealed environment. After the bacteriophage-exposed sample has been incubated, it may then be applied to the flow strip without transfer to a separate applicator. Further, using either the bulb 72 or the flexible bulb 672, the application to the flow strip can be accurately controlled, again without the use of a separate applicator instrument.

Another related feature of the invention is that the detection and identification assemblies 200, 400 are reliable, accurate, and easy to use by conventional health care workers without specialized training. Further, unlike other bacteriophage bacteria identification systems, no laboratory or expensive equipment is required. The accuracy and reliability of the result is not strongly affected by the amount of sample applied to the sample areas 282, 284, 494. The unique formulation of the broth 11, 12, 671, 911 provides latitude for the process, permitting accuracy and reliability even with some deviation from optimum conditions and handling. Simple directions and labeling 15, 17, 23, 50, 52, 60, 240, 244, 246, 248, 250, etc., easily understandable by conventional health care workers, are written directly on the apparatus. The flow strips are easily read and include a control test that allows confirmation of the working state of the system, again by unspecialized health care workers.

It is a feature of the invention that the conjugate 281, 881 is either located in the sample pad 282, 882 or mixed with the sample prior to the application to the flow strip. That is, in the prior art, the conjugate was located in a separate conjugate area, such as 283, 883 on the flow strip. However, it has been found that conjugating the bacteriophage earlier results in significantly improved reliability and accuracy of the tests. Several embodiments in which the conjugate is located in the sample pad have been discussed above. The alternative in which the conjugate is mixed with the bacteriophage prior to application to the sample pad and flow strip is discussed below.

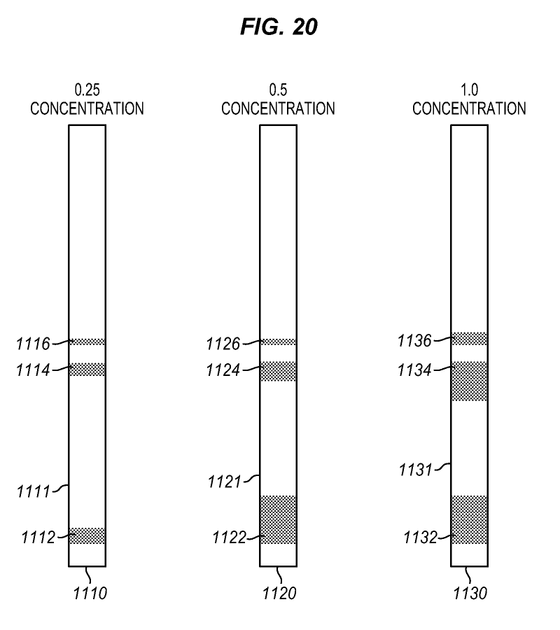

It is a feature of the invention that the conjugate and bacteriophage may be mixed prior to application to the flow strip, and the conjugate areas 283, 285, 492, 883 may be eliminated. A conjugate is anything that can bind with the bacteriophage to assist in the detection assay. Well-known conjugates include antibodies, antibodies conjugated to a marker such as a colored bead, an enzyme, a colloidal particle such as gold, and biotin which, when attached to a bacteriophage, can permit the bacteriophage to become attached to a streptavidin-coated object. In the example of FIG. 20, the conjugate was a gold conjugated Rabbit bacteriophage antibody. The conjugate may be included in the fluid 11, 12, 671, 911 or may be placed in the incubation container 101, 606, 993, by including it in a reagent element, such as bead 122 or discs 123 and 622.

FIG. 20 depicts three photographs showing results of a preferred embodiment of the invention in which the conjugate and bacteriophage are mixed prior to application to a porous flow strip. Because the United States Patent and Trademark Office does not accept gray scale in drawings, it is not possible to exactly depict the photographs. Thus, in the depictions of FIG. 20, the thickness of the “line” represents both the intensity and thickness of the corresponding line in the photographs. The demonstration was run at three different concentrations of the conjugate. The wider the line, the wider and more intense was the corresponding line in the photograph. The comparative thickness and intensity was estimated visually, so these depictions are not intended to be exact reproductions. In the preferred process, the sample mixture was applied to the end 1119, 1120, and 1130 of half “dip sticks” 1111, 1121, 1131, respectively. These half-dipsticks were lateral flow strips as described above with the applicator pad cut off. The sample was applied to the opposite end of the strip from the end that included the applicator pad simply because that worked. Each flow strip included two lines of embedded antibodies: a control line 1114, 1124, and 1134, which was a line of an antibody to a Rabbit antibody, and a test line 1116, 1126, and 1136, which was a line of a Rabbit antibody to the bacteriophage, i.e., an antibody to the MP112 bacteriophage. The samples were prepared as follows. In each case, gold conjugated Rabbit antibody to the MP112 bacteriophage was added to a tube containing the bacteriophage in a base media. The bacteriophage was the bacteriophage which had generated the antibody. In each case, the tube contained 2.5×106 pfu/mL (plaque forming units per milliliter) of the bacteriophage and the base media was a solution of TSP (tryptic soy broth), 27 mmol/L (millimoles/Liter) of sodium pyruvate, and lauric acid at a concentration of 20 μg/mL. See the above for a more detailed description of the base media. After adding the conjugated antibody, 3.3 μL-5 μL of JMI 105 staphylococcus aureus from JMI Laboratories 345 Beaver Creek Centre, Suite A, North Liberty, Iowa 52317 was spiked into the tube with a pipette. The sample was incubated for four hours and then 100 μL was applied to the end 1110, 1120, and 1130, respectively, of a half-dipstick 1111, 1121 and 1131, respectively, and chased with 100 μL chase buffer. In each of the three samples, the concentration of conjugate was different. In the sample of strips 1111, 1121, and 1131, the concentration had an optical density of 0.25, 0.5, and 1.0, respectively. As can be seen from FIG. 20, in each of the strips 1111, 1121, and 1131, both the control lines, i.e., lines 1114, 1124, and 1134, respectively, and the test lines, i.e., lines 1116, 1126, and 1136, developed. The test lines 1116 and 1126 at 0.25 and 0.5 concentration, respectively, were weak, and under some conditions might not be able to be relied on for a definitive test. However, test line 1136 for the 1.0 concentration was clear and would reliably indicate a positive test under any conditions. The conjugate at 0.25 optical density concentration and bacteriophage with no bacteria was also applied, under the above conditions, to a control strip; and the conjugate only, again at 0.25 optical density concentration with no bacteriophage, was applied under the above conditions to a second control strip. The control line developed in each control strip, and the test line was not discernible.

As a further test of the effect of adding the gold conjugated antibody to the sample at the same time as the phage, the samples that were applied to strips 1111, 1121, and 1131 were also applied to a standard plate. The optical density (OD) 0.25 gold conjugate had no effect on the plaque numbers. That is, the plates were cleared. In the case of the OD 0.5 sample, the plate had 500 plaques yielding a bacteriophage concentration of 5×108 pfu/mL. In the case of the OD 1.0 sample, the plate had 42 plaques yielding a bacteriophage concentration of 4.2×107 pfu/mL. As can be seen from FIG. 20, though the action was reduced, it was still detectable on a half-dipstick.

The above-described results indicate that the lateral flow strip method is feasible with a process in which the conjugate is added prior to application to the porous flow strip. At the same time, a surprising result that might be more significant was also found. In each of the strips 1111, 1121, and 1131, a visible line 1112, 1122, and 1132 appeared just above the end of the dipstick to which the sample was applied. This line also faintly appeared in the control with the phage and conjugate but was much lighter. There was some sign of this line in the conjugate only control, but it was too light to be certain. It is noted that these visible lines were not generated by an antibody line. It is believed that what occurred to produce these lines was that the agglutinated complexes of phage, colloidal gold, and bacteria were formed and were being filtered out by the fabric of the flow strip as the sample flowed along the length of the strip. These results indicate that a test could be developed that depended only on the filtering of the complexes, rather than on the fixation of the complexes to a line of antibodies. This would produce a simpler and more economical test.

The various embodiments of collectors, incubator-applicators, and test strips described above may be combined in any way, and the combinations shown are just by way of example. For example, the collector assembly 530 may be combined with incubator-applicator assembly 120, and collector assembly 100 may be combined with incubator applicator assembly 720. Any of the flow strip assemblies, 200, 400, or any of the flow strips 208, 209, or 410, may be used with any combination of collector assemblies and incubator-applicator assemblies. After reviewing this disclosure, those skilled in the art will understand that many other variations of these parts and assemblies may be designed and used in many different combinations.

There has been described microorganism identification and antibiotic susceptibility/resistance apparatus and methods which are sensitive, simple, fast, and/or economical, and having numerous novel features. It should be understood that the particular embodiments described within this specification are for purposes of example and should not be construed to limit the invention, which will be described in the claims below. Further, it is evident that those skilled in the art may now make numerous uses and modifications of the specific embodiments described without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by the microorganism identification apparatus and methods described.

What is claimed as new and desired to be protected by Letters Patent of the United States is:

1. A method of determining the presence or absence of a target bacterium in a test sample, said method comprising:
combining a bacteriophage specific to said target bacteria with said sample;
incubating said sample sufficiently to permit said bacteriophage to infect said target bacteria and to multiply in said target bacteria to create progeny bacteriophage to create a bacteriophage exposed sample;
providing a sample pad and a porous strip, said sample pad in contact with said porous strip, said porous strip having fixation substance embedded in said porous strip at a fixation area, said fixation substance capable of attaching to either said bacteriophage or a bacteriophage conjugate;
applying said progeny bacteriophage to said sample pad;
conjugating said progeny bacteriophage to a bacteriophage conjugate to form a bacteriophage/conjugate complex;
flowing said bacteriophage/conjugate through said porous strip to said fixation area; and
determining the presence or absence of a sufficient amount of fixated bacteriophage/conjugate complex at said fixation area to determine the presence or absence of said target bacterium.

2. A method as in claim 1 wherein said sample pad contains said conjugate, and said conjugating is performed in said sample pad.

3. A method as in claim 1 wherein said conjugating is performed prior to said applying.

4. A method as in claim 1 wherein said conjugating comprises combining said conjugate and said progeny bacteriophage in an incubation container.

5. A method as in claim 1 wherein said porous strip includes a conjugate area, and said conjugating is performed in said test strip.

6. A method as in claim 1 wherein said providing a porous strip comprises embedding an antibody in said fixation area.

7. A method as in claim 1 wherein said providing comprises embedding a control fixation substance in a control area in said porous strip, said control fixation substance comprising a substance capable of attaching to said conjugate.

8. A system for determining the presence or absence of a target bacterium in a test sample, said system comprising:
a bacteriophage specific to said bacterium;
a detectable conjugate to said bacteriophage;
a sample pad for receiving a test sample that may contain said target bacterium; and
a porous strip in contact with said sample pad and having a fixation area comprising a test fixation substance embedded in said porous strip, said test fixation substance comprising a substance capable of attaching to either said bacteriophage or said conjugate.

9. A system as in claim 8 wherein said conjugate is embedded in said sample pad.

10. A system as in claim 8, and further including a bacteriophage incubation container containing said conjugate.

11. A system as in claim 8 wherein said system also includes a strip holder enclosing said porous strip, said strip holder having a window exposing said fixation area and, labeling adjacent said window indicating the approximate location of said fixation area.

12. A system as in claim 8 wherein said test fixation substance comprises an antibody to said bacteriophage.

13. A system as in claim 8 wherein said porous strip further includes a conjugate area in which said conjugate is embedded, said conjugate area located in said porous strip between said sample pad and said fixation area.

14. A system as in claim 8, and further comprising a control area comprising a control fixation substance, said control fixation substance comprising a substance capable of attaching to said conjugate.

15. A system as in claim 8 wherein said bacteriophage comprises a plurality of different types of bacteriophage, each of said different bacteriophage being specific to said target bacterium.

16. A kit for determining the presence or absence of a target bacterium in a sample to be tested, said kit comprising:
a bacteriophage;
a bacteriophage incubation container having an opening for inserting a sample containing said target bacterium; and
a substrate at least a portion of which changes color if a predetermined amount of either said bacteriophage or a biological substance associated with said bacteriophage is present.

17. A kit as in claim 16, and further including a bacteriophage container containing said bacteriophage in a buffer solution.

18. A kit as in claim 17, and further including a connector for connecting said bacteriophage container to said incubation container with a fluid tight seal.

19. A kit as in claim 17, and further including a buffer solution in said bacteriophage container, said buffer solution also containing a substance that enhances bacteriophage amplification, said substance being different than said buffer solution.

20. A kit as in claim 19 wherein said buffer solution also contains a substance that inhibits replication of said bacteriophage in potentially cross-reactive, non-target bacteria.

21. A kit as in claim 16 wherein said substrate comprises a porous strip.

22. A kit as in claim 21, and further including a dropper for applying a fluid containing said target bacterium and said bacteriophage to said porous strip.

23. A kit as in claim 17 wherein said bacteriophage container contains a conjugate for said bacteriophage.

24. A kit as in claim 16 wherein said incubation container contains a conjugate for said bacteriophage.

25. A kit as in claim 16 wherein said bacteriophage comprises a plurality of different types of bacteriophage, each of said different bacteriophage being specific to said target bacterium.

26. A bacteriophage incubator comprising a bacteriophage incubation container and a bacteriophage incubation fluid container containing bacteriophage incubation fluid comprising bacteriophage; said bacteriophage incubator characterized by:
said incubation container and said incubation fluid container each having a connection portion formed to permit said incubation container to be connected to said incubation fluid container with a fluid tight seal;
said incubator further comprising a valve located between said incubation fluid container and said incubation container when said incubation fluid container is connected to said incubation container, said valve having a closed condition in which said incubation fluid container does not fluidly communicate with said incubation container and an open position in which said fluid container fluidly communicates with said incubation container.

27. A method of determining the presence or absence of a target bacterium in a sample to be tested, said method comprising:
combining with said sample an amount of parent bacteriophage capable of attaching to said target bacteria to create a bacteriophage-exposed sample;
providing conditions to said bacteriophage-exposed sample sufficient to allow said bacteriophage to attach to said target bacteria to provide a detectable amount of either bacteriophage or a biological substance associated with said bacteriophage in a bacteriophage-exposed sample;
and assaying said bacteriophage-exposed sample to detect the presence or absence of said bacteriophage or said biological substance associated with said bacteriophage to determine the presence or absence of said target bacteria, said method characterized by said combining comprising:
providing a collection/incubation system comprising a bacteriophage container containing a fluid comprising said bacteriophage and an incubation container;
collecting said bacteria with a collector;
placing said collector in said incubating container;
connecting said bacteriophage container to said incubation container, said connecting providing a fluid tight seal; and
without breaking said fluid tight seal, opening said valve to permit said bacteriophage fluid to flow into said incubation container.

28. A method of determining the resistance or susceptibility of a target bacterium to an antibiotic, said method comprising:
(a) providing a first sample containing said target bacteria;
(b) adding said antibiotic to said first sample;
(c) combining said first sample with a bacteriophage capable of infecting said target bacteria to create a first bacteriophage-exposed sample;
(d) providing conditions to said first bacteriophage-exposed sample sufficient to allow said bacteriophage to infect said target bacterium and to multiply in said target bacterium to create a detectable amount of either said bacteriophage or a biological substance associated with said bacteriophage in said first bacteriophage-exposed sample;
(e) assaying said first bacteriophage-exposed sample to detect the presence or absence of an amount of said bacteriophage or said biological substance associated with said bacteriophage; and
(f) if said amount of said bacteriophage or biological substance is above an amount associated with a predetermined indicator, determining that said target bacteria is resistant to said antibiotic; and if said amount of said bacteriophage or biological substance associated with said bacteriophage is below a predetermined susceptibility indicator, determining that said target bacteria is susceptible to said antibiotic.
